(19) United States
(12) Patent Application Publication
(10) Pub. No.: US 2014/0073611 A1
(43) Pub. Date: Mar. 13, 2014

(54) METHODS FOR DRUG SCREEN USING ZEBRAFISH MODEL AND THE COMPOUNDS SCREENED THEREFROM

(71) Applicant: National Taiwan University, Taipei (TW)

(72) Inventors: I-Jong Wang, Taipei City (TW); Wei-Ting Ho, New Taipei City (TW); Ting-Hsuan Chiang, Taipei City (TW); I-Tsen Lin, Taipei City (TW)

(73) Assignee: National Taiwan University, Taipei (TW)

(21) Appl. No.: 13/899,297

(22) Filed: May 21, 2013

Related U.S. Application Data
(60) Provisional application No. 61/649,611, filed on May 21, 2012.

(51) Int. Cl. A61K 31/65; A61K 31/4402; A61K 31/4045; A61K 31/164; A61K 31/381; A61K 31/5377; A61K 31/18; A61K 31/357; A61K 31/4166; A61K 31/541; A61K 31/351; A61K 31/445; A61K 31/353; A61K 31/4439; A61K 31/138; A61K 31/416; A61K 31/185; A61K 31/198; A61K 31/401; A61K 31/4178; A61K 31/05; A61K 31/366; A61K 31/165

(52) U.S. Cl. CPC A61K 31/65; A61K 31/165; A61K 31/4402; A61K 31/4045; A61K 31/164; A61K 31/381; A61K 31/5377; A61K 31/18; A61K 31/357; A61K 31/4166; A61K 31/541; A61K 31/351; A61K 31/445; A61K 31/353; A61K 31/4439; A61K 31/138; A61K 31/416; A61K 31/185; A61K 31/198; A61K 31/401; A61K 31/4178; A61K 31/05; A61K 31/366
USPC 514/152; 514/616; 514/352; 514/419; 514/445; 514/233.5; 514/602; 514/467; 514/389; 514/227.8; 514/459; 514/327; 514/456; 514/338; 514/648; 514/403; 514/665; 514/562; 514/423; 514/381; 514/731; 514/460

(57) ABSTRACT

The disclosure relates to a platform of using zebrafish in screening candidates for treating and/or preventing myopia and keratoconus disease. The disclosure is mainly based on that Lumican, one of several SLRPs, plays an important role in the regulation of fibrillogenesis or the genes affecting the size of eyeballs in zebrafish, in addition to playing an important role in clinical myopia. Therefore, the disclosure uses the established zebrafish model to further identify the drugs affecting the expression of lumican and collagen fibrillogenesis, and/or the regulation of eyeball size. These drugs are potential candidates for treating myopia and/or keratoconus disease.

### CROSS-REFERENCE TO RELATED APPLICATION(S)

This application claims priority to U.S. Provisional Patent Application No. 61/649,611, filed on May 21, 2012, the disclosure of which is hereby incorporated by reference in its entirety.

### FIELD OF THE DISCLOSURE

The present disclosure relates to a method for drug screening using zebrafish as a model. Particularly, the disclosure relates to a method for identifying candidate compounds for affecting the expression of lumican and collagen fibrillogenesis and for treating a disease medicated by expression of lumican and/or collagen fibrillogenesis and the candidate compounds identified therefrom. More particularly, the method identifies drugs for treating and/or preventing myopia and/or keratoconus disease.

### BACKGROUND OF THE DISCLOSURE

Myopia is the most common eye disorder in the world. In Western countries, the prevalence of myopia is about 16%-27%, whereas in Asian countries it may be much higher. For example, there is an 82% prevalence of myopia for the Chinese population in Singapore. A refractive error equal to or below −6 diopters (D), defined as high myopia, is also termed “pathological myopia” because its potential complications, including cataracts, glaucoma, macular degeneration, and retinal detachment, might lead to blindness. Genetic and environmental factors may cause myopia. It has been estimated that about half or more of all persons suffering from myopia have axial myopia caused by elongation of the eye along the visual axis. At birth, the human eye is about two-thirds the size of an adult eye and is relatively short in the axial direction. As a consequence, young children tend to be farsighted. As the eye grows during childhood, compensatory fine tuning of the optical properties of the cornea and lens occurs, increasing the ocular length. Often the entire process takes place virtually flawlessly and the eye becomes emmetropic. When this fine tuning process fails, however, it usually brings about a lengthened eye. As a result, distant images get focused in front of the plane of the retina and axial myopia results. In clinical trials, only anti-cholinergic drugs (such as atropine) have been used to control the progress of myopia. However, the long-term use of atropine needed to control the progress of myopia can cause side effects such as blurred vision, constipation, decreased sweating, difficulty sleeping, dizziness, drowsiness, dry mouth, nose, or skin, headache, loss of appetite, loss of taste, nausea, or nervousness. Therefore, there is a need to develop replacement drugs for controlling, preventing and/or treating myopia.

Thinning of the sclera, particularly at the posterior pole, is a crucial feature of the development of high myopia in humans. In primates, the sclera is a fibrous extracellular matrix (ECM) composed of collagens (mainly Type I collagen), elastin, proteoglycans and other components that are arranged in lamellae produced by scleral fibroblasts. (Alex Gentle et al., The Journal of Biological Chemistry, 2003, Vol. 278, No. 19, pp. 16587-16594) Scleral remodeling involves the regulation of numerous gene products such as collagens, proteoglycans, matrix metalloproteinases (MMPs), and tissue inhibitors of metalloproteinases (TIMPs), including smaller diameter collagen fibrils, reduced glycosaminoglycan (GAG) content, reduced proteoglycan (Decorin) synthesis, and increased MMP-2. Selective changes in mRNA levels also have been found for some proteins, including collagen I, MMP-2, MT1-MMP, TIMP-3, and TGF-β, suggesting that retina-derived signals modulate scleral gene expression to remodel the scleral tissue and modulate scleral creep rate. (John T. Siegwart Jr and Thomas T. Norton, Invest Ophthalmol Vis Sci. 2002 July; 43(7): 2067-2075.) Scleral remodeling is intrinsic to myopia progression, and these biochemical changes are actually a precursor to changes in the biomechanical properties of the sclera, and ultimately to the development of myopia. The adult human sclera contains three major proteoglycans: aggrecan, biglycan, and decorin, which contribute to the structural properties of the sclera. The ratios of these proteoglycans will change with the condition of the sclera. Decorin and biglycan belong to a class of small leucine-rich proteoglycans (SLRPs), which also includes lumican, DSPG-3 (dermatan sulfate proteoglycan 3, PG-L epiphycan), fibromodulin, PRELP (proline-arginine-rich and leucin-rich repeat protein), keratocan, chondroadherin, and osteoglycin. Decorin, biglycan, lumican, and fibromodulin bind to type I collagen and influence matrix assembly and organization. Animal studies reveal that the proteoglycan synthesis rate significantly influences eyeball growth and myopia development. The synthesis rate of decorin in the sclera of marmosets is inversely correlated with vitreous chamber elongation rates. Biglycan and lumican mRNA levels were lowered in the sclera during experimentally induced myopia and increased during recovery. Lumican, a member of small leucine-rich proteoglycan (SLRP) family, is one of the major extracellular components in interstitial collagenous matrices of the corneal stroma, aorta, skin skeletal muscle, lung, kidney, bone, cartilage, and intervertebral discs, etc.

In corneal tissue, lumican contains keratin sulfate chains present as a proteoglycan, whereas in non-corneal tissues, lumican is present as a low or non-sulfated glycoprotein (50-57 kDa). Its wide distribution implies that lumican has multiple functions regarding tissue morphogenesis and maintenance of tissue homeostasis. This was best illustrated by the multiple clinical manifestations observed in lumican knockout mice, which exhibited corneal opacity, skin and tendon fragility, delayed wound healing, and low fertility. Indeed, lumican has been shown to play essential roles in corneal transparency by regulating collagen fibrillogenesis in wound healing by modulating epithelial cell migration, and in the epithelium-mesnchyme transition of the injured lens. Lumican deficient mice and Lum(−/−)Fmod(−/−) mice showed collagen fibril diameter alteration and features of high myopia, suggesting that these proteoglycans play an important role in the biomechanical properties of sclera. In addition, linkage studies of high myopia identified potential loci MYP1 (Xq28) and MYP3 (12q21-23), located near or containing several SLRP genes, including biglycan (Xq27ter), decorin (12q21-22), lumican (12q21.3-22), and DSPG3 (12q21). MYP3 may be also responsible for 25% of autosomal dominant high myopia in families in the U.K. Therefore, candidate genes relevant to myopia development that map to MYP3, including decorin, lumican and DSPG3, are of great interest. More recently, novel 14 mutations in SLRP genes have been associated with high myopia; for example, c.893-105G>A in the LUM gene might have a protective role or be in linkage disequilibrium with a protective allele.

The zebrafish is a popular vertebrate model to study biology and the molecular genetics of development. Zebrafish can be easily managed (3-4 cm length as an adult) in large numbers in the laboratory. The ability to combine embryological and genetic methodology has established the zebrafish as a powerful research tool. Transparent embryos allow fundamental vertebrate developmental processes from gastrulation to organogenesis. In addition, the eye, heart beats, and blood circulation of the embryo can be readily and easily observed. Touch, sight and behavioral responses can also be monitored in live embryos under the dissecting microscope. Several features, such as a short generation time of 3-4 months, make zebrafish particularly suitable for genetic studies. Most previous studies that included a number of eye mutations produced with the chemical mutagen ENU reported a reduced eye size, disorganized retina, cyclopia, reduced ganglion cell layer and loss of photoreceptors. Lung-Kun Yeh has isolated and characterized the zebrafish keratocan and lumican gene and found an increased eyeball size after knockdown of zebrafish lumican during their development, which is compatible with and relevant to the clinical findings in children myopia. In children myopia, there were similar findings of axial elongation of eyeballs in children who had an alteration of SNP at the human lumican gene promoter. Decreased zebrafish lumican promoter activity was suspected to have been related to this SNP. It was proposed that lumican, one of a number of small leucine rich polypeptides, plays an important role in the regulation of fibrillogenesis and eye development which possibly affect the size of eyeball. (Lung-Kun Yeh et al., Journal of Biological Chemistry, 2010, Vol. 285, No. 36, pp. 28141-28155.) This prior reference also indicates that down-regulation of zlum expression by antisense zlum morpholinos manifested ocular enlargement resembling axial myopia due to disruption of the collagen fibril arrangement in the sclera and resulted in scleral thinning, and that administration of muscarinic receptor antagonists, e.g. atropine and pirenzepine, effectively subdued the ocular enlargement caused by morpholinos in in vivo zebrafish larvae assays. Therefore, this prior reference suggests that zebrafish can be used as an in vivo model for screening myopia-treating compounds.

However, there is still a need to further explore applications of the zebrafish model in achieving practical myopia drug screening and finding effective drugs for controlling, preventing and/or treating myopia.

### SUMMARY OF THE DISCLOSURE

The present disclosure provides a method of using zebrafish with big eye to identify a candidate compound that can be used to affect the expression of lumican and/or collagen fibrillogenesis, and/or treat myopia and/or keratoconus disease, the method comprises contacting a test compound with the zebrafish with big eye and identifying the test compound as a candidate compound if a ratio of the big eye in the zebrafish decreases.

The disclosure also provides a method of using lumican gene and/or collagen fibrillogenesis-relating gene knockdown zebrafish to identify a candidate compound that can be used to affect the expression of lumican and/or collagen fibrillogenesis, and/or treat myopia and/or keratoconus disease, the method comprises contacting a test compound with lumican gene and/or collagen fibrillogenesis-relating gene knockdown zebrafish, determining the number of the big eye in the zebrafish and identifying the test compound as a candidate compound if a ratio of the big eye in the zebrafish decreases.

### BRIEF DESCRIPTION OF THE DRAWING

The file of this patent application contains at least one drawing executed in color. Copies of this patent with color drawing(s) will be provided by the Patent and Trademark Office upon request and payment of the necessary fee.

FIG. 1 depicts a series of morphological changes of zLum knockdown fish in 3-7 dpf, in accordance with one embodiment of the disclosure;

FIG. 2 depicts the effect of lumican gene knockdown on eye size, in accordance with one embodiment of the disclosure;

FIGS. 3(A)-3(H) depict zLum-MO knockdown-induced ultrastructural changes in a corneal stroma (CS), an anterior sclera (AS), and a posterior sclera (PS), in accordance with one embodiment of the disclosure, in which

FIG. 3A depicts WT fish at 12 dpf stage in toludine blue staining and indicates corneal stroma (CS), anterior sclera (AS), and posterior sclera (PS),

FIG. 3B depicts the diameters of collagen fibrils of corneal stroma, anterior, and posterior sclera in the 12 dpf old wild-type and zLum MO injected groups,

FIG. 3C depicts collagen fibril architecture in the corneal stroma of WT fish,

FIG. 3D depicts collagen fibril architecture in the corneal stroma of zLum MO injected fish,

FIG. 3E depicts collagen fibril architecture in the anterior sclera of WT fish,

FIG. 3F depicts collagen fibril architecture in the anterior sclera of zLum MO injected fish,

FIG. 3G depicts collagen fibril architecture in the posterior sclera of WT fish, and

FIG. 3H depicts collagen fibril architecture in the posterior sclera of zLum MO injected fish;

FIG. 4A depicts ultrastructural changes in scleral thinning in the zLum-MO group, in accordance with one embodiment of the present disclosure, in which the top is adjacent to the retina and two to three layers of scleral fibroblastic cells with collagen fibril formation between the layers are found at the posterior sclera of the WT fish at 7 dpf stage;

FIG. 4B depicts ultrastructural changes in scleral thinning in the zLum-MO group, in accordance with one embodiment of the present disclosure, in which the top is adjacent to the retina and only one to two layers of fibroblastic cells appear at the posterior sclera of the zLum-MO-injected fish at 7 dpf stage;

FIG. 4C depicts ultrastructural changes in scleral thinning in the zLum-MO group, in accordance with one embodiment of the present disclosure, in which scleral thinning is observed obviously in the zLum-MO-injected fish at 7 dpf stage;

FIG. 5 depicts zLum expression in the zebrafish 44 embryo 2˜4 days postfertilization, in accordance with one embodiment of the present disclosure;

FIG. 6 depicts western blot (upper portion) and mRNA rescue analyses (lower portion), in accordance with one embodiment of the present disclosure;

FIG. 7A depicts the outer margin of the retinal pigmented epithelium layer (RPE(red color)) and the diameter of the scleral coat (D(green color)) in zebrafish in accordance with one embodiment of the present disclosure;

FIG. 7B depicts the outer margin of the retinal pigmented epithelium layer (RPE(red color)) and the diameter of the scleral coat (D(green color)) in zebrafish in accordance with one embodiment of the present disclosure;

FIG. 7C depicts a chart showing axial changes in fish in accordance with one embodiment of the present disclosure;

FIG. 7D depicts a chart showing changes the ratio of RPE/scleral coat in fish in accordance with one embodiment of the present disclosure;

FIG. 8A depicts phenotype of WT fish at 7 dpf stage in accordance with one embodiment of the present disclosure;

FIG. 8B depicts normal phenotype of RS MO injected embryos at 7 dpf stage in accordance with one embodiment of the present disclosure;

FIG. 8C depicts phenotype of zLum MO injected embryos at 7 dpf stage in accordance with one embodiment of the present disclosure;

FIG. 8D depicts phenotype of atropine-treated, zLum MO injected embryos at 7 dpf stage in accordance with one embodiment of the present disclosure;

FIG. 8E depicts phenotype of pirenzepine-treated, zLum MO injected embryos at 7 dpf stage in accordance with one embodiment of the present disclosure;

FIG. 8F depicts phenotype of methoctramine-treated, zLum MO injected embryos at 7 dpf stage in accordance with one embodiment of the present disclosure;

FIG. 9 depicts phenotype (left) and expression patterns (right) of atropine rescues zLum knockdown morphant, in accordance with one embodiment of the present disclosure;

FIG. 10 depicts the big eye ratios of the zebrafish treated with marimastat, doxycycline, captopril, minocycline hydrochloride, atropine, aspirin, propofol and N-acetylcysteine, in accordance with one embodiment of the present disclosure;

FIG. 11A depicts big eye ratios of zebrafish treated with tetracycline, in accordance with one embodiment of the present disclosure;

FIG. 11B depicts big eye ratios of zebrafish treated with minocycline, in accordance with one embodiment of the present disclosure;

FIG. 11C depicts big eye ratios of zebrafish treated with doxycycline, in accordance with one embodiment of the present disclosure;

FIG. 11D depicts big eye ratios of zebrafish treated with marimastat, in accordance with one embodiment of the present disclosure; and

FIG. 11E depicts big eye ratios of zebrafish treated with batimastat, in accordance with one embodiment of the present disclosure.

### DETAILED DESCRIPTION OF THE DISCLOSURE

The disclosure describes a platform for using zebrafish to screen candidates for treating and/or preventing myopia and keratoconus disease. The disclosure found that lumican, one of a number of SLRPs, plays an important role in the regulation of fibrillogenesis and genes that influence the size of eyeballs in zebrafish, in addition to playing an important role in clinical myopia. Therefore, the disclosure uses an established zebrafish model to further identify the drugs affecting the expression of lumican and collagen fibrillogenesis, and the regulation of eyeball size. These drugs are potential candidates of treating myopia and keratoconus disease, including but not limiting to, metalloprotease (MMP) inhibitors, TGF-beta inhibitors, anticholinergic or muscarinic compounds and COX inhibitors.

As used in the specification and claims, the singular form “a”, “an”, and “the” include their plural references unless the context clearly dictates otherwise. For example, the term “a cell” includes a plurality of cells, including mixtures thereof.

As used herein, “expression” refers to the process by which a polynucleotide is transcribed into mRNA and/or the process by which the transcribed mRNA (also referred to as “transcript”) is subsequently translated into peptides, polypeptides, or proteins.

A “control” is an alternative subject or sample used in an experiment for comparison purposes.

The terms “test compound” and “candidate compound” refer to any chemical entity, pharmaceutical, drug, or the like that is a candidate for being used to achieve the utility mentioned herein, such increasing the expression of lumican and collagen fibrillogenesis, and/or treating or preventing myopia and/or keratoconus disease. Test compounds comprise both known and potential therapeutic compounds. A test compound can be determined to be therapeutic through the screening methods of the present disclosure.

The term “big eye” denotes that an eye with a value of the axial length of the retinal pigmented epithelium layer divided by the axial length of the scleral coat is less than 0.7.

The term “treat” and “treatment” mean cause, or the act of causing, a postponement of development of a disorder and/or a reduction in the severity of symptoms that will or are expected to develop. The terms further include ameliorating existing symptoms or preventing symptoms.

The term “therapeutically effective amount” means that amount of a drug or pharmaceutical agent that will elicit the biological or medical response of a tissue system, animal or human that is being sought by a researcher or clinician, resulting in a beneficial effect for at least a statistically significant fraction of patients, such as a improvement of symptoms, a cure, a reduction in disease load.

The term “subject” is intended to include living organisms susceptible to conditions or diseases, disease states or conditions as generally disclosed, but not limited to, throughout this specification. Examples of subjects include humans, dogs, cats, cows, goats, and mice. The term subject is further intended to include transgenic species.

The term “alkyl” as used herein means a saturated straight chain or branched non-cyclic hydrocarbon having an indicated number of carbon atoms (e.g., C₁-C₂₀, C₁-C₁₀, C₁-C₈, C₁-C₆, C₁-C₄, etc.). Representative saturated straight chain alkyls include -methyl, -ethyl, -n-propyl, -n-butyl, -n-pentyl, -n-hexyl, -n-heptyl, -n-octyl, -n-nonyl and -n-decyl; while representative saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

The term “alkenyl” by itself or as part of another substituent, as used herein, refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6) alkenyl.

The term “alkynyl” by itself or as part of another substituent, as used herein, refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6) alkynyl.

The term “aryl” by itself or as part of another substituent, as used herein, refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15) aryl, with (C5-C10) being even more preferred.

The term “heteroaryl” by itself or as part of another substituent, as used herein, refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, .beta.-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

The term “pharmaceutically acceptable salts and prodrugs” as used herein refers to those carboxylate salts, acid addition salts or base addition salts, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use of the compounds of the disclosure. The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

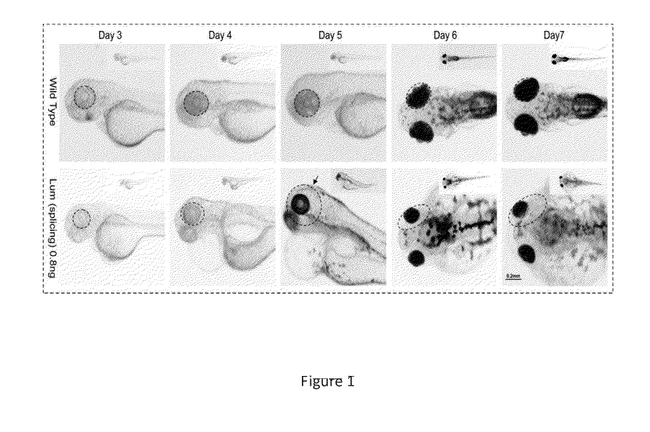

FIG. 1 depicts a series of morphological changes of zLum knockdown fish in 3-7 dpf, in accordance with one embodiment of the disclosure. Coupled with eye development, zLum KD fish causes the sclera enlargement in progress. On Day 5, retinal detachment 110 can be clearly observed through the microscope.

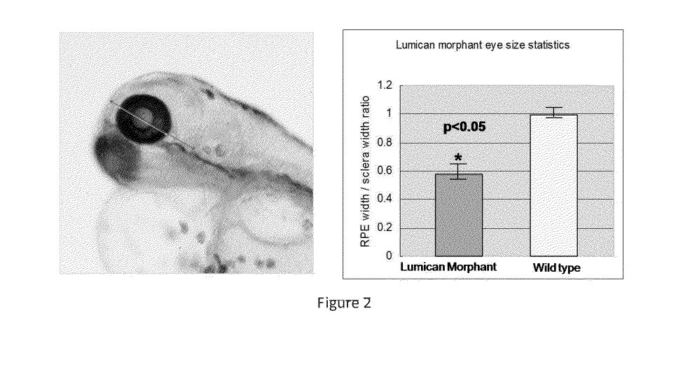

FIG. 2 depicts the effect of lumican gene knockdown on eye size. As shown in FIG. 2, morphometric measurements of sclera width are depicted in red lines 210 and RPE width are depicted in white lines 212. Results of comparing Lumican morphant with wild type in RPE width/sclera width ratio show knockdown of lumican due to ectasia of the sclera leading to axial elongation.

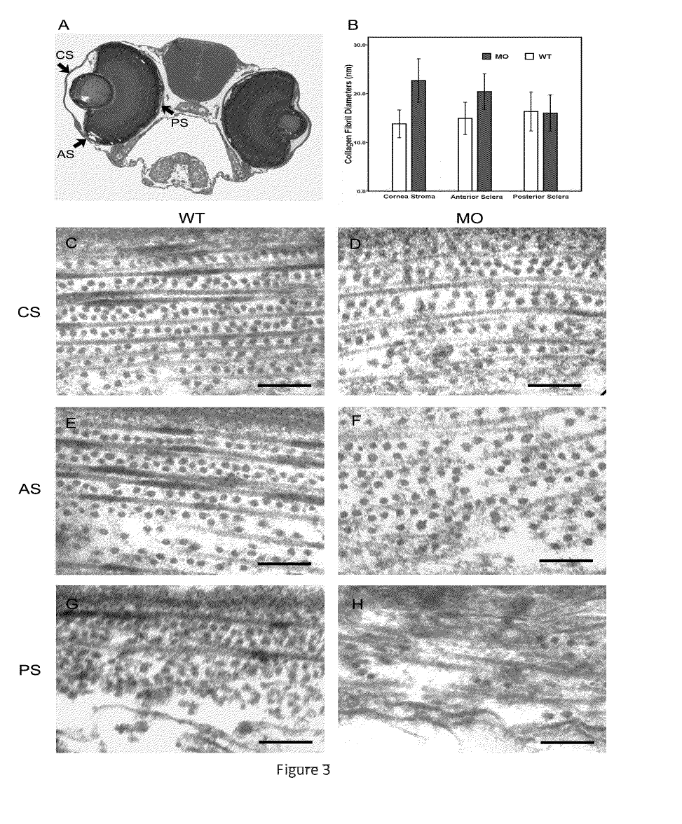

FIGS. 3(A)-(H) depict zLum-MO knockdown induces ultrastructural changes in a corneal stroma (CS) 310, an anterior sclera (AS) 312, and a posterior sclera (PS) 314. FIG. 3(A) depicts WT fish at 12 dpf stage in toludine blue staining. The figure indicates corneal stroma (CS) 310, anterior sclera (AS) 312, and posterior sclera (PS) 314. FIG. 3(B) depicts the diameters of collagen fibril are analyzed in the corneal stroma, anterior and posterior sclera of the 12 dpf-old wild type WT 316 and zLum-MO-injected 318 groups. Significant increases in the collagen fibril diameter of corneal stroma 310 and anterior sclera 312 are noted in the zLum-MO group, whereas the diameter of collagen fibril in the posterior sclera 314 in both groups is not significantly different. FIGS. 3(C)-(H) depict morphological comparison of collagen fibril architecture in the corneal stroma in FIGS. 3(C) and (D), anterior scleral tissue in FIGS. 3(E) and (F), and posterior scleral tissue in FIGS. 3(G) and (H) between the control group FIGS. 3(C, E, G) and zLum-MO-injected group FIGS. 3(D, F, H) at the 12 dpf stage. FIG. 3(C) depicts a TEM micrograph showing regular and smaller fibril architecture of collagen localized in the corneal stroma of the wild type group. FIG. 3(D) depicts an irregular arrangement and increased collagen fibril diameter is found in the corneal stroma of the zLum-MO-injected group. FIG. 3(E) depicts a TEM micrograph showing relatively regular fibril architecture of collagen localized in the anterior sclera of the wild type group. FIG. 3(F) depicts irregular collagen fibrils with increased fibril diameter are noted in the anterior sclera of the zLum-MO-injected group. FIG. 3(G) depicts a top adjacent to the retina. TEM micrograph shows fibril architecture of collagen localized in the posterior sclera of the wild type group. FIG. 3(H) depicts a top adjacent to the retina. TEM micrograph shows irregular collagen fibril architecture in the posterior sclera of the zLum-MO-injected group. In FIGS. 3(C)-(H), the scale bar represents 100 nm.

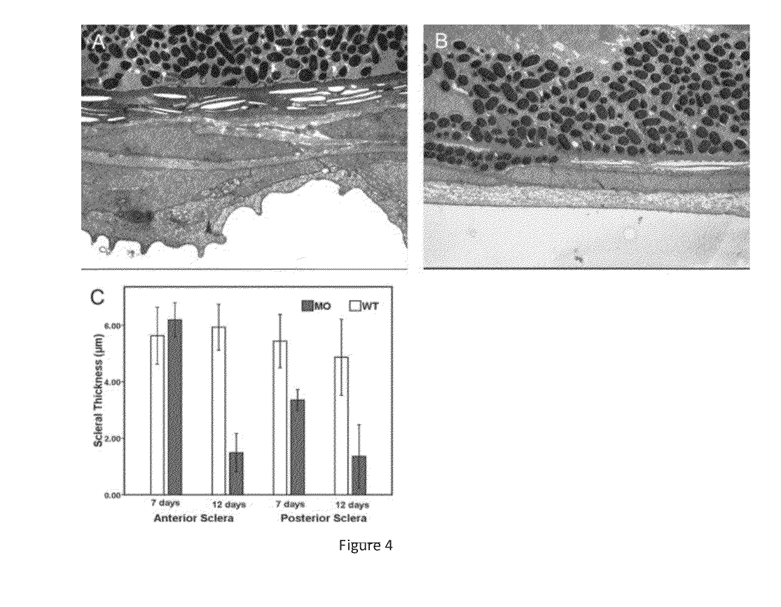

FIGS. 4(A)-(C) depict ultrastructural changes in scleral thinning in the zLum-MO group. In FIG. 4(A), the top is adjacent to the retina. Two to three layers of scleral fibroblastic cells with collagen fibril formation between the layers found at the posterior sclera of the WT fish at 7 dpf stage. In. FIG. 4(B), the top is adjacent to the retina. Only one to two layers of fibroblastic cells at the posterior sclera of the zLum-MO-injected fish at 7 dpf stage. In FIG. 3(C), scleral thinning is observed obviously in the zLum-MO-injected fish at 7 dpf stage. The phenomenon is much more prominent in the zLum-MO-injected fish at 12 dpf stage. In particular, significant scleral thinning is observed in the posterior sclera of the zLum-MO-injected fish at 7 and 12 dpf stage as compared with the wild type group. As depicted in FIGS. 4(A) and (B), the scale bar represents 1.5 um.

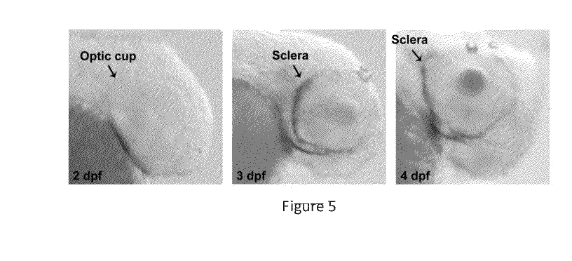

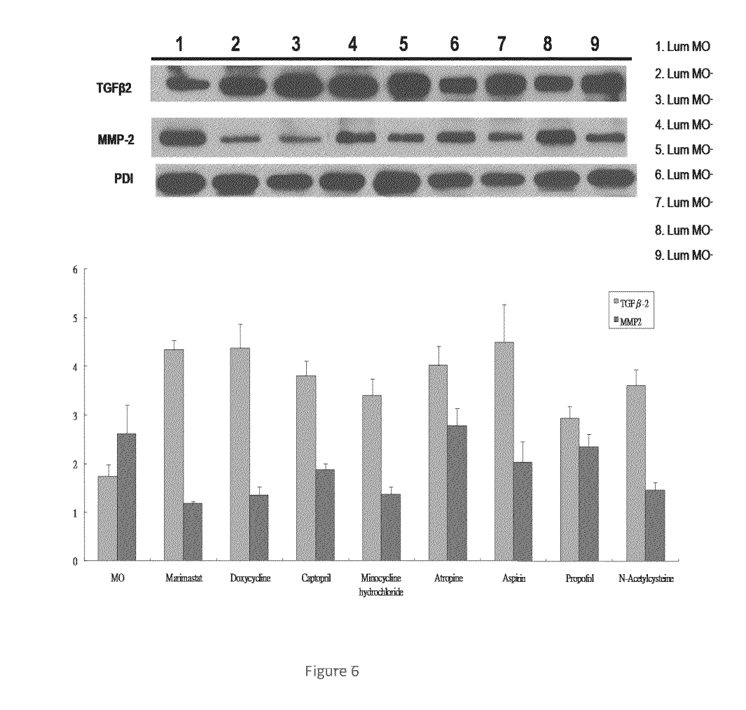

FIG. 5 depicts zLum expression in the zebrafish 44 embryo 2˜4 days postfertilization. zLum mRNA is expressed specifically in the sclera of zebrafish since 3 dpf by whole mount in situ hybridization.

FIG. 6 depicts western blot and mRNA rescue analyses. As shown in FIG. 6, the left-hand columns depict, in the western blot analysis, the lumican, collagen 1a1, TGF-beta, and TIMP2 decreased in the lumican morphant. In contrast, the MMP2 expression increased, as shown in the right-hand columns. The abnormally large eyes could be rescued with lumican and collagen 1a1 mRNAs. However, they could be also be rescued with ppih, hsp 47 and rx1 mRNAs, which are related to collagen fibrillar formation and eye development, respectively.

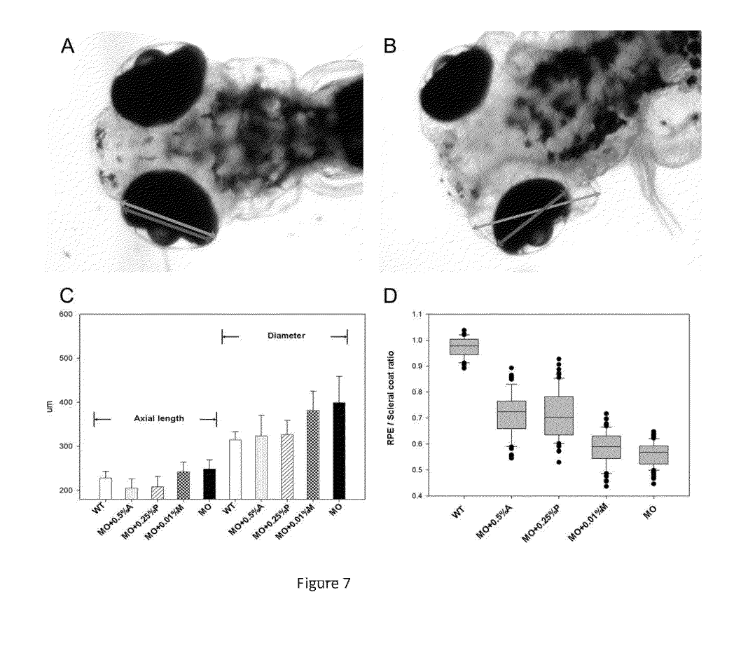

FIGS. 7(A)-(D) depict a zebrafish drug screen assay. FIGS. 7(A) and (B) illustrate and define the outer margin of the retinal pigmented epithelium layer (RPE(red color)) and the diameter of the scleral coat (D(green color)) in zebrafish. FIG. 7(C) depicts significant decreases in excessive axial elongation in the zLum-MO-injected fish at the 7 dpf stage after being treated with 0.5% atropine(A) and 0.25% pirenzepine(P), whereas there were no obvious changes in excessive axial elongation after being treated with 0.01% methoctramine(M), as shown in lane 1: WT; lane 2: MO+ 0.5% A; lane 3: MO+ 0.25% P; lane 4: MO+ 0.01% M; lane 5: MO. Significant decreases in the diameter of the scleral coat of the zLum-MO-injected fish at the 7 dpf stage after being treated with 0.5% atropine and 0.25% pirenzepine, whereas there were no obvious changes in the zLum-MO-injected group treated with 0.01% methoctramine, as shown in lane 6: WT; lane 7: MO+ 0.5% A; lane 8: MO+ 0.25% P; lane 9: MO+0.01% M; lane 10: MO. FIG. 7(D) depicts significant decreases in the ratio of RPE/scleral coat (%) were noted during ocular enlargement developed due to the reduction of zLum protein. Some muscarinic receptor antagonists (atropine and pirenzepine) attenuate the decreasing ratio of the RPE/scleral coat due to the reduction of zLum protein, whereas there are no obvious changes in the decreased ratio of the RPE/scleral coat in the methoctramine-treated group, as shown in lane 1: WT; lane 2: MO+ 0.5% A; lane 3: MO+ 0.25% P; lane 4: MO+ 0.01% M; lane 5: MO.

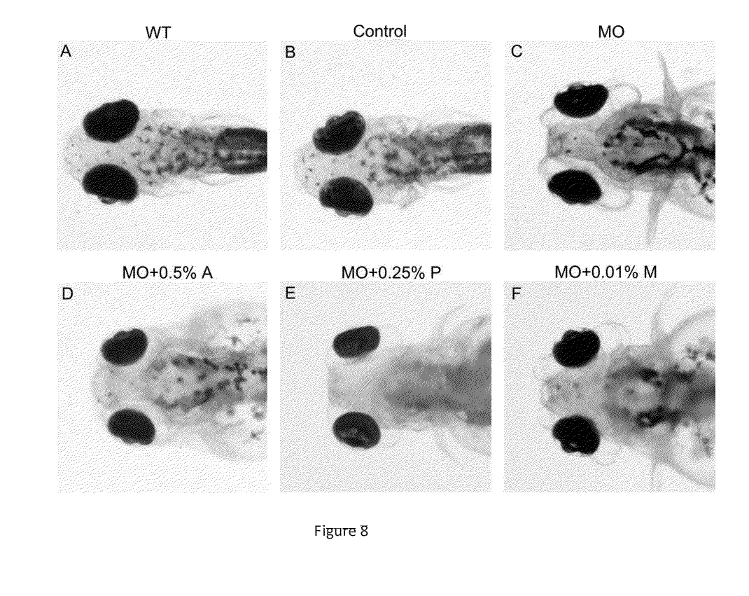

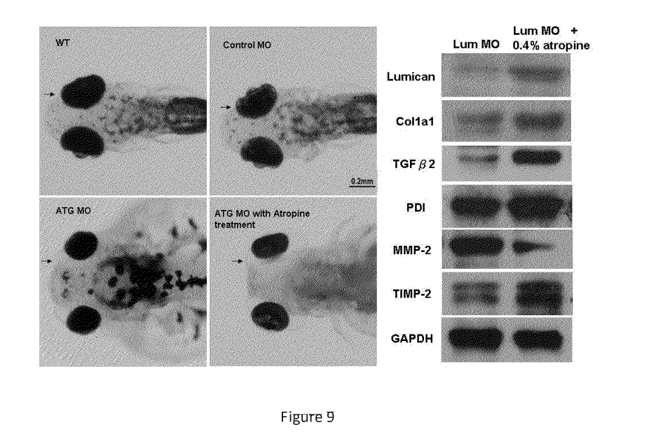

FIGS. 8(A)-(F) depict normal phenotype of WT fish at 7 dpf stage (FIG. 8(A)); normal phenotype of RS-MO-injected embryos at 7 dpf stage (FIG. 8(B)); significantly enlarged eyeball of zlum-MO-injected fish at 7 dpf stage (FIG. 8(C)); significant decreases in ocular enlargement was noted in the zlum-MO-injected larvae at 7 dpf stage after being treated with 0.5% atropine for 2 days (FIG. 8(D)); decreases in ocular enlargement were also found in the zLum-MO-injected larvae at 7 dpf stage after being treated with 0.25% pirenzepine for 2 days (FIG. 8(E)); and no obvious changes in the phenotypes of zlum-MO-injected fish at 7 dpf stage after being treated with 0.01% methoctramine (FIG. 8(F)).

FIG. 9 depicts atropine rescues zLum knockdown morphant. It can reverse the expressions of lumican, collagen 1a1, TGF-beta, MMP2 and TIMP2 which decreased in lumican morphant with atropine.

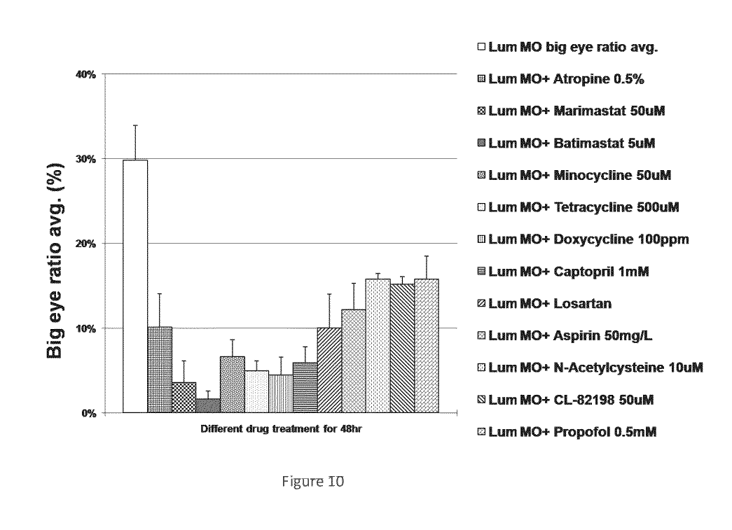

FIG. 10 depicts the big eye ratios of the zebrafish treated with marimastat, doxycycline, captopril, minocycline hydrochloride, atropine, aspirin, propofol and N-acetylcysteine.

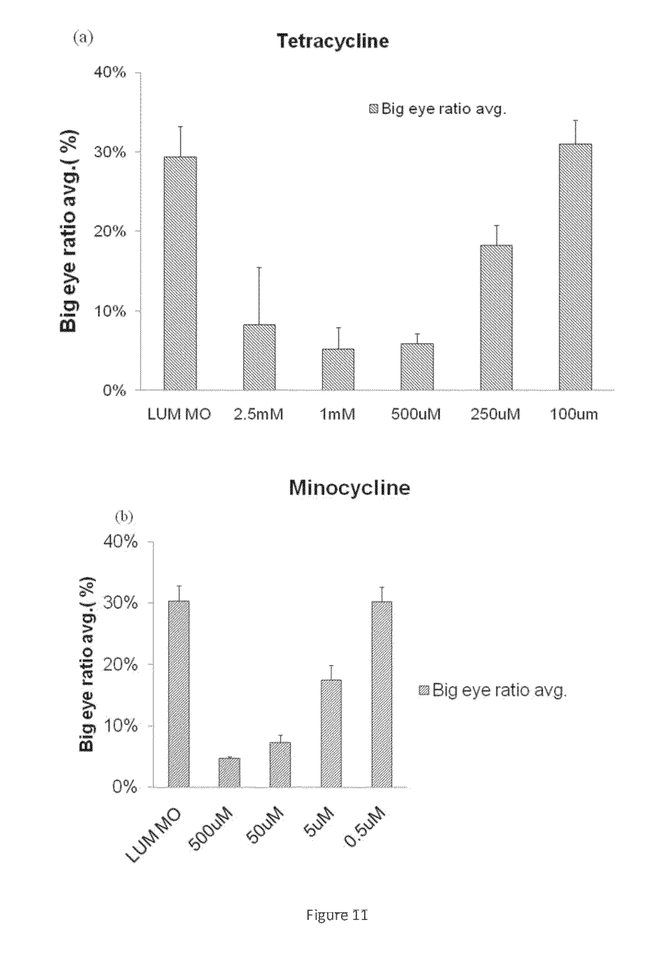

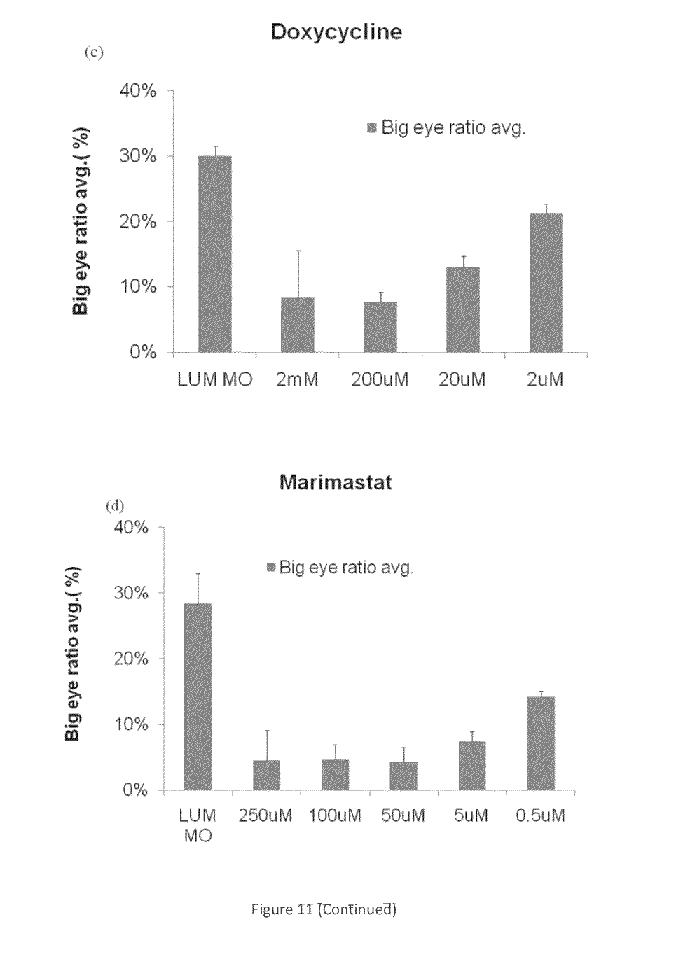

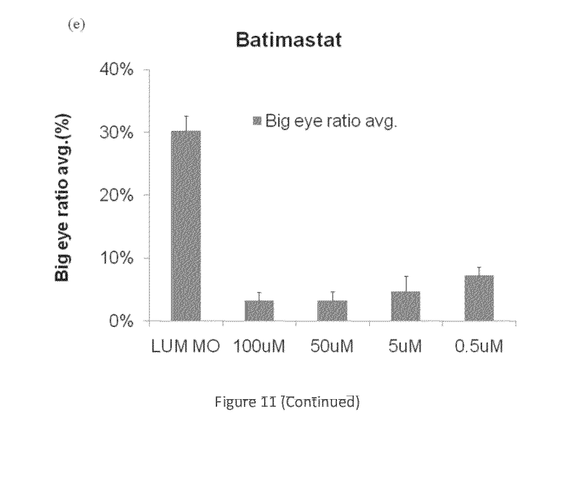

FIGS. 11(a)-(e) depict the big eye ratios of the zebrafish treated with tetracycline (FIG. 11(a)), minocycline (FIG. 11(b)), doxycycline (FIG. 11(c)), marimastat (FIG. 11(d)) and batimastat (FIG. 11(e)).

In one aspect, the disclosure provides a method of using zebrafish with big eye to identify a candidate compound that can be used to affect the expression of lumican and/or collagen fibrillogenesis, and/or treat myopia and/or keratoconus disease, the method comprises contacting a test compound with the zebrafish with big eye and identifying the test compound as a candidate compound if a ratio of the big eye in the zebrafish decreases. In one embodiment, the test compound is identified as a candidate compound if the ratio of the big eye in the zebrafish decreases relative to the total number of the big eyes in the control zebrafish that is not treated with the test compound.

In another aspect, the disclosure provides a method of using lumican gene and/or collagen fibrillogenesis-relating gene knockdown zebrafish to identify a candidate compound that can be used to affect the expression of lumican and/or collagen fibrillogenesis, and/or treat myopia and/or keratoconus disease, the method comprises contacting a test compound with lumican gene and/or collagen fibrillogenesis-relating gene knockdown zebrafish, determining the number of the big eye in the zebrafish and identifying the test compound as a candidate compound if a ratio of the big eye in the zebrafish decreases. In one embodiment, the test compound is identified as a candidate compound if the ratio of the big eye in the zebrafish decreases, relative to the total number of the eyes in the zebrafishes or that of the big eyes in the control zebrafish that is not treated with the test compound.

In one embodiment, the disclosure provides a method of identifying a candidate compound affecting the expression of lumican and collagen fibrillogenesis and/or the regulation of eyeball size, comprising:
(a) introducing an antisense mRNA of lumican gene and/or collagen fibrillogenesis-relating gene or an analog of the antisense mRNA into plural fertilized embryos of zebrafish;
(b) exposing the zebrafish obtained from (a) to a test compound for a sufficient length of time and then collecting the zebrafish; and
(c) determining the number of the big eye in the zebrafish and identifying the test compound as a candidate compound if a ratio of the big eye in the zebrafish decreases.

Preferably, the antisense mRNA in (a) are lumican or keratocan antisense mRNA. Preferably, the knockdown zebrafish in (b) are exposed to the test compound at their optic cup formation. Preferably, the resulting zebrafish in (b) are collected at their cornea establishment stage. Preferably, the test compound in (c) is identified as a candidate compound if the ratio of the big eye in the zebrafish decreases, relative to the total number of the eyes of the zebrafish or the total number of the big eyes in the control zebrafish. In view of the above, the method comprises the following steps:
(a) introducing an antisense mRNA of lumican gene and/or collagen fibrillogenesis-relating gene or an analog of the antisense mRNA into plural fertilized embryos of zebrafish;
(b) exposing the zebrafish obtained from (a) to a test compound for a sufficient length of time and then collecting the zebrafish; and
(c) determining the number of the big eye in the zebrafish and identifying the test compound as a candidate compound if a ratio of the big eye in the zebrafish decreases relative to the total number of the eyes in the zebrafishes or that of the big eyes in the control zebrafish that is not treated with the test compound.

In another embodiment, the disclosure provides a method of identifying a candidate compound treating and/or preventing myopia and/or keratoconus disease, comprising:
(a) introducing an antisense mRNA of lumican gene and/or collagen fibrillogenesis-relating gene or an analog of the antisense mRNA into plural fertilized embryos of zebrafish;
(b) exposing the zebrafish obtained from (a) to a test compound for a sufficient length of time and then collecting the zebrafish; and
(c) determining the number of the big eye in the zebrafish and identifying the test compound as a candidate compound if a ratio of the big eye in the zebrafish decreases.

Preferably, the antisense mRNA in (a) are lumican or keratocan antisense mRNA. Preferably, the knockdown zebrafish in (b) are exposed to the test compound at their optic cup formation. Preferably, the resulting zebrafish in (b) are collected at their cornea establishment stage. Preferably, the test compound in (c) is identified as a candidate compound if the ratio of the big eye in the zebrafish decreases, relative to the total number of the eyes of the zebrafish or the total number of the big eyes in the control zebrafish. In view of the above, the method comprises the following steps:
(a) introducing an antisense mRNA of lumican gene and/or collagen fibrillogenesis-relating gene or an analog of the antisense mRNA into plural fertilized embryos of zebrafish;
(b) exposing the zebrafish obtained from (a) to a test compound for a sufficient length of time and then collecting the zebrafish; and
(c) determining the number of the big eye in the zebrafish and identifying the test compound as a candidate compound if a ratio of the big eye in the zebrafish decreases relative to the total number of the eyes in the zebrafishes or that of the big eyes in the control zebrafish that is not treated with the test compound.

The screening assays described herein provide methods for identifying compounds that affect the expression of lumican and collagen fibrillogenesis and the regulation of eyeball size, and treat and/or prevent myopia and keratoconus disease using the decrease of the ratio of enlarged eyeballs in lumincan knockdown zebrafish as an indicator of compounds that affect the expression of lumican and collagen fibrillogenesis and treat and/or prevent myopia and keratoconus disease.

Compounds identified in the assays described herein are candidate compounds that can be used (i) to affect the expression of lumican and collagen fibrillogenesis and the regulation of eyeball size and/or (ii) as lead compounds to develop related compounds that can be used to treat and/or prevent myopia and keratoconus disease.

### Keratocan and Lumican Genes in Zebrafish

Lumican, one of several SLRPs, plays an important role in the regulation of fibrillogenesis or the genes affecting the size of eyeballs in zebrafish, in addition to playing an important role in clinical myopia. Similar to keratocan and lumican genes of human and mice, zebrafish keratocan and lumican genes have all the structural features of SLRPs, i.e. a central domain of leucine-rich repeats flanked by N- and C-terminal domains with conserved cysteines. The size and structure of the zebrafish keratocan and lumican genes are similar to the mammalian keratocan and lumican genes. Interestingly, both the zebrafish lumican and keratocan genes have been mapped to the same genome. In addition, similar to the mammalian keratocan and lumican genes, the zebrafish keratocan and lumican genes are TATA-less genes. Also, the most striking difference between keratocan and lumican expression in the corneas of zebrafish and mammal is that they are expressed mainly in the corneal epithelial layer in the case of the former instead of the stromal layer (keratocytes). It is also a very promising field to explore in developmental biology.

### Knockdown Zebrafish

Surprisingly, an increased size of eyeball (i.e., big eye) was associated with the knockdown of zebrafish lumican, keratocan and/or collagen fibrillogenesis-relating gene(s) during the development of zebrafish, a clinical manifestation similar to clinical findings in children myopia. In children myopia, axial elongation of eyeballs in children was correlated with an alteration of SNP at the human lumican gene promoter in patients. Decreased expression level of zebrafish lumican, keratocan and/or collagen fibrillogenesis-relating gene(s) by knockdown using an antisense or its analog may mimic the molecular mechanism causing the axial elongation observed in patients with this SNP. According to the disclosure, an antisense mRNA of lumican is introduced into a fertilized embryo of zebrafish to obtain a lumican knockdown zebrafish. In one embodiment, the lumican antisense is morpholino. Preferably, the morpholino has the sequence: 5′-GATCCCAGAGCAAACATGGCTGCAC-3′.

### Exposure of Knockdown Zebrafish to a Test Compound and Collection of the Resulting Zebrafish

External development and optical clarity during embryogenesis allow for visual analyses of early developmental processes, and high fecundity and short generation times facilitate genetic analyses. The adult zebrafish eye is emmetropic, and it is able to transmit both visible and ultraviolet wavelengths, evidenced by adult responsiveness to ultraviolet wavelengths. The development of the zebrafish eye is similar to eye development in other species of fish and mammals. It begins with the optic primordial at about 12 h postfertilization (hpf). By 24 hpf, the eyecups are well developed, and by about 30 hpf, ganglion cells are found in a small area of the ventronasal retina. At 50 hpf, the retinal layers become apparent across parts of the retina. Young zebrafish are hyperopic and become emmetropic by 72 hpf, which is the same time the extraocular muscles appear to be adult-like, and the optokinetic response is evident.

According to the disclosure, the knockdown zebrafish are exposed to a test compound at the optic cup formation stage thereof. Generally, at around 24 hours after fertilization, the optic cup of zebrafish is formed. The knockdown zebrafish can be exposed to the test compound at the optic cup formation stage. After contacting the knockdown zebrafish with the test compound, the test compound, if it is a potential candidate, may start to activate the expression of lumican and collagen fibrillogenesis, thereby decreasing the enlargement of eyeball and treating and/or preventing myopia and/or keratoconus disease. Retina lenses are established around 48 hours after fertilization, and the sclera and cornea are established around 72 hours after fertilization. At sclera and cornea establishment stage, the zebrafish are collected.

### Determination of Big Eye Zebrafish and Identification of Candidate Compounds

Big eye in a zebrafish is an indicator of myopia. As used herein, a “big eye” refers to an eye with an enlarged axial length of eyeball and denotes the value of the axial length of the retinal pigmented epithelium layer divided by the axial length of the scleral coat less than 0.7. The axial length of a retinal pigmented epithelium layer and the axial length of a scleral coat can be measured by any method known in the art—for example, dissecting microscopy.

The test compound can be identified as a candidate compound affecting the expression of lumican and/or collagen fibrillogenesis and/or regulation of eyeball size, and/or treating and/or preventing myopia and/or keratoconus disease if the ratio of the number of big eyes decreases relative to the total number of eyes in zebrafish or that of big eyes in control zebrafish that is not contacted with the test compound. Preferably, the test compound can be identified as a candidate compound if the ratio of the number of big eyes is less than 30% relative to the total number of eyes in zebrafish or that of big eyes in control zebrafish that is not contacted with the test compound. Preferably, the ratio is less than 15%. More preferably, the ratio decreases to about 0% to about 30%, about 0% to about 25%, about 0% to about 20%, about 0% to about 15%, about 0% to about 10%, about 1% to about 30%, about 1% to about 25%, about 1% to about 20% or about 1% to about 15%.

In a further aspect, screening of test compounds is accomplished by identifying those in a group of test compounds that decrease the ratio of the big eye of zebrafish to less than 30%. Test compounds that decrease the big eye ratio are also referred to herein as “candidate compounds.”

The screening methods of the disclosure can be used to identify compounds, e.g., small organic or inorganic molecules (molecular weight less than 1,000 Da), oligopeptides, oligonucleotides, or carbohydrates, that decrease the big eye ratio of the lumican knockdown zebrafish. As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include metalloprotease inhibitors, collagenase inhibitors, TGF-β pathway activators, TGF-β inhibitors and Cox inhibitors.

Metalloprotease Inhibitors for Use in a Method for Affecting the Expression of Lumican and/or Collagen Fibrillogenesis, and/or Treating Myopia and/or Keratoconus Disease

In one embodiment, the disclosure provides a method for treating a disease medicated by expression of lumican and/or collagen fibrillogenesis and/or treating myopia and/or keratoconus disease, comprising administering to the subject a therapeutically effective amount of a MMP inhibitor.

Metalloproteases (MMPs) are also thought to play a major role on cell behaviors such as cell proliferation, migration (adhesion/dispersion), differentiation, angiogenesis, apoptosis, and host defense. Inhibitors of metalloproteases are known. Examples include natural biochemicals such as tissue inhibitors of metalloproteinases (TIMPs), a2-macroglobulin and their analogs or derivatives. A number of smaller peptide-like compounds that inhibit metalloproteases have been described. Thiol group-containing amide or peptidyl amide-based metalloprotease (MMP) inhibitors are known as is shown in, for example, WO95/12389, WO96/11209 and U.S. Pat. No. 4,595,700. Hydroxamate group-containing MMP inhibitors are disclosed in a number of published patent applications such as WO 95/29892, WO 97/24117, WO 97/49679 and EP 0 780 386 that disclose carbon back-boned compounds, and WO 90/05719, WO 93/20047, WO 95/09841 and WO 96/06074 that disclose hydroxamates that have a peptidyl back-bones or peptidomimetic back-bones. In addition, other pyrimidine-based MMP inhibitors, hydroxypyrone-based MMP inhibitors, phosphorous-based MMP inhibitors and tetracycline-based MMP inhibitors have also been reported (Cancer Metastasis Rev., 2006, 25:115-136.)

According to one embodiment of the disclosure, the MMP inhibitor is a peptidomimetic hydroxamate MMP inhibitor having the following Formula (I) or a pharmaceutically acceptable salt, prodrug, solvate, stereoisomer or enantiomer thereof,

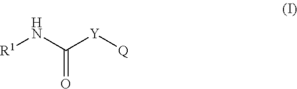

wherein

Q is absent or

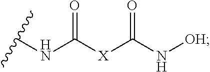

X is C₁₋₁₀ alkylene, C₂₋₁₀ alkenylene or C₂₋₁₀ alkynylene, unsubstituted or substituted by one or more OH, C₁₋₁₀ straight or branched alkyl, C₂₋₁₀ straight or branched alkenyl, C₁₋₁₀alkylC₅₋₁₅aryl, C₁₋₁₀alkenylC₅₋₁₅aryl, C₁₋₁₀alkynylC₅₋₁₅aryl, C₁₋₁₀alkylsulfanylC₅₋₁₅ aryl, C₁₋₁₀alkylsulfonylC₅₋₁₅aryl, C₁₋₁₀alkylsulfinylC₅₋₁₅aryl, C₁₋₁₀alkyloxy or C₅₋₁₅aryl;
Y is C₁₋₁₀ alkylene, C₂₋₁₀ alkenylene or C₂₋₁₀ alkynylene, unsubstituted or substituted by one or more OH, C₁₋₁₀ straight or branched alkyl, C₂₋₁₀ straight or branched alkenyl, C₁₋₁₀alkylC₅₋₁₅aryl, C₁₋₁₀alkenylC₅₋₁₅aryl, C₁₋₁₀alkynylC₅₋₁₅aryl, C₁₋₁₀alkylsulfanylC₅₋₁₅aryl, C₁₋₁₀alkylsulfonylC₅₋₁₅aryl, C₁₋₁₀alkylsulfinylC₅₋₁₅aryl, C₁₋₁₀alkyloxy, C₅₋₁₅aryl, C₁₋₁₀alkylC₅₋₁₅aryl, C₅₋₁₄heteroaryl, C₁₋₁₀alkylC₅₋₁₄heteroaryl, or C₁₋₁₀alkylsulfanylC₅₋₁₄heteroaryl, provided that when Q is absent, Y is C₅₋₁₄heteroaryl; wherein the heteroaryl is optionally substituted and has 1 to 3 heteroatoms independently selected from N, O and S; and
R¹ is H, OH, C₁₋₁₀alkyl, C₂₋₁₀alkenyl, C₂₋₁₀alkynyl, C₅₋₁₅aryl, C₁₋₁₀alkylC₅₋₁₅aryl, C₅₋₁₄heteroaryl, or C₁₋₁₀alkylC₅₋₁₄heteroaryl.

Preferably, when Q is absent, Y is

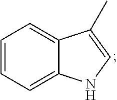

more preferably, when Q is absent, Y is

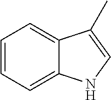

and R₁ is C₅₋₁₅heteroaryl; most preferably, when Q is absent, Y is

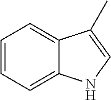

### and R₁ is

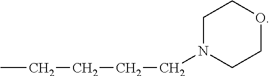

Preferably, when Q is

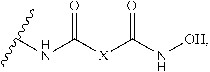

X is —CH₂—, —CH(CH₂CH₂(CH₃)₂)—, or —CH₂CH₂—, —CH(CH₂CH₂(CH₃)₂)CH(CH₃)—, —CH(CH₂CH₂(CH₃)₂)CH(CH₂—S-phenyl)-, —CH(CH₂CH₂(CH₃)₂)CH(OCH₃)—, —CH(CH₂CH₂(CH₃)₂)—, or —CH₂CH₂—, —CH(CH₂CH₂(CH₃)₂)CH(CH₃)—, —CH(CH₂CH₂(CH₃)₂)CH₂—, —CH(CH₂CH₂(CH₃)₂)CH(OH)—, or —CH(CH₂CH₂(CH₃)₂)CH(CH₂—S-thienyl)-; Y is —CH(CH₂—phenyl)-, —CH(C(CH₃)₃)— or —CH(CH₂-indolyl)-; and R₁ is CH₃ or phenyl.

More preferably, the compound of Formula (I) is selected from the group consisting of:

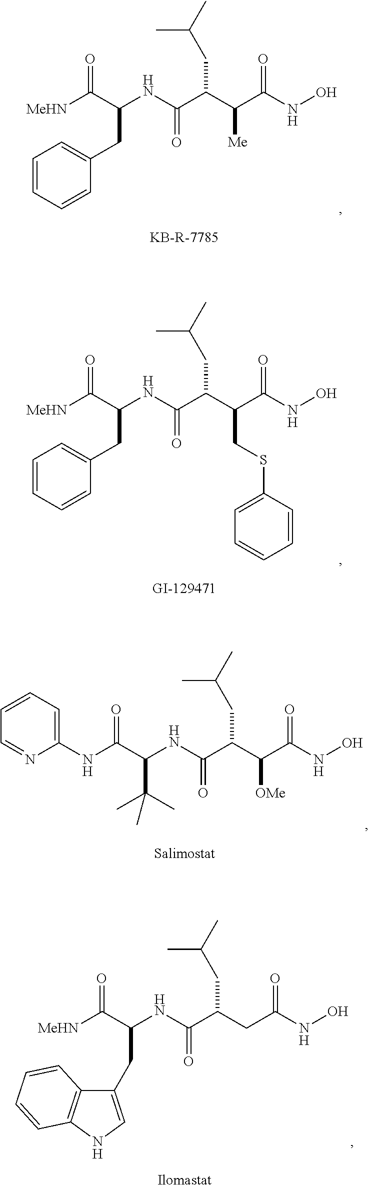

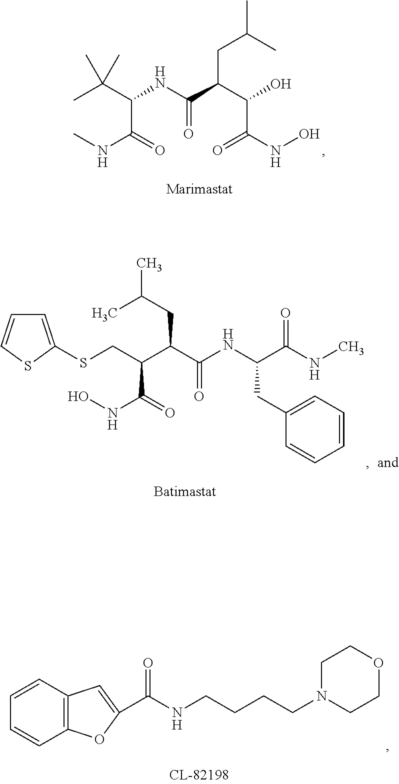 [SMILES: CNC(=O)[C@@H](NC(=O)[C@@H](CC(C)C)[C@H](O)C(=O)NO)C(C)(C)C ; CNC(=O)[C@H](Cc1ccccc1)NC(=O)[C@H](CC(C)C)[C@H](CSc1cccs1)C(=O)NO ; O=C(NCCCCN1CCOCC1)c1cc2ccccc2o1]

or a pharmaceutically acceptable salt, prodrug, solvate, stereoisomer or enantiomer thereof.

According to another embodiment of the disclosure, the MMP inhibitor is a tetracyclic-based MMP inhibitor having the following Formula (II) or a tautomer or pharmaceutically acceptable salt, prodrug or solvate thereof,

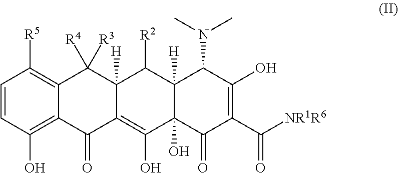

wherein
R¹ and R⁶ are each independently H, C₁₋₁₀alkylC₅₋₁₄heteroaryl, or C₁₋₁₀NR⁷R⁸;
R² is hydrogen or OH;
R³ and R⁴ are each independently H, OH, NH₂, NO, CN, C₁₋₁₀alkyl, C₁₋₁₀alkenyl or C₁₋₁₀alkynyl;
R⁵ is hydrogen, halogen, NH₂, OH, NO, CN, C₁₋₁₀ alkyl, NHC₁₋₁₀alkyl, N(C₁₋₁₀alkyl)₂, C₅₋₁₅aryl or C₅₋₁₄heteroaryl; and
R⁷ and R⁸ are each independently H, C₁₋₁₀alkyl C₁₋₁₀alkylNH₂COOH or taken together with the nitrogen atom to which each is attached form a 3 to 8 membered heteroaryl;
   wherein heteroaryl has 1 to 3 heteroatoms independently selected from N, O and S.

Preferably, R¹ is H; R⁶ is H, —CH₂-pyrrolyl, —CH₂—NH—CH₂—CH₂—CH₂—CH₂—CH(NH2)-COOH; R² is H or oxo; R³ is H or OH; R⁴ is H or OH and R⁵ is NH₂, N(CH₃)₂ or halogen.

More preferably, the compound of Formula (II) is selected from the group consisting of:

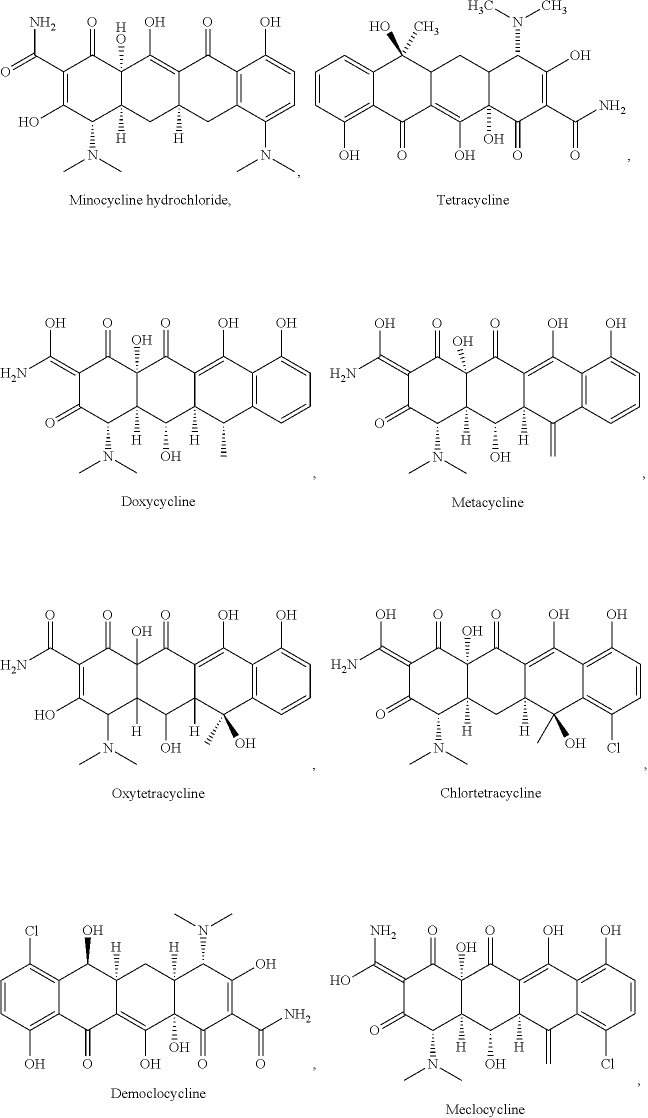

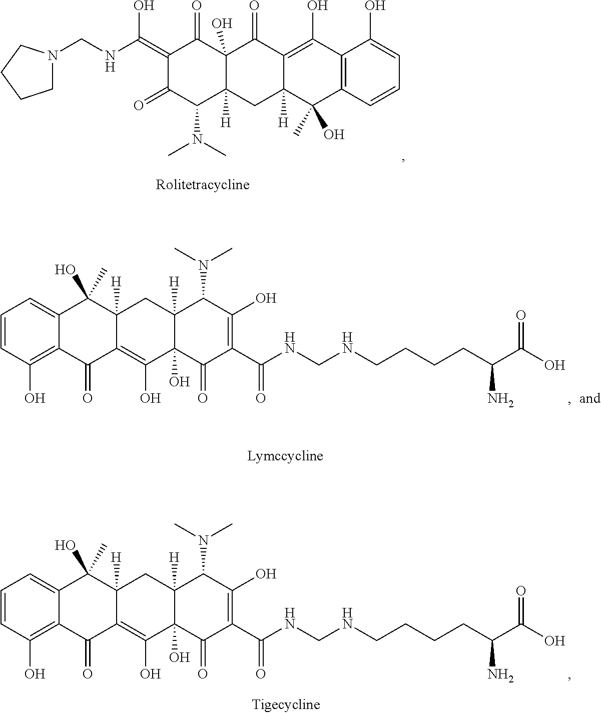 [SMILES: CN(C)[C@@H]1C(=O)/C(=C(/O)NCN2CCCC2)C(=O)[C@@]2(O)C(=O)C3=C(O)c4c(O)cccc4[C@@](C)(O)[C@H]3C[C@@H]12 ; CN(C)[C@@H]1C(O)=C(C(=O)NCNCCCC[C@H](N)C(=O)O)C(=O)[C@@]2(O)C(O)=C3C(=O)c4c(O)cccc4[C@@](C)(O)[C@H]3C[C@@H]12 ; CN(C)[C@@H]1C(O)=C(C(=O)NCNCCCC[C@H](N)C(=O)O)C(=O)[C@@]2(O)C(O)=C3C(=O)c4c(O)cccc4[C@@](C)(O)[C@H]3C[C@@H]12]

or a tautomer or pharmaceutically acceptable salt, prodrug or solvate thereof.

According to another embodiment of the disclosure, the MMP inhibitor is a diaryl ether hydroxamate having the following Formula (III) or a pharmaceutically acceptable salt, prodrug, solvate, stereoisomer or enantiomer thereof,

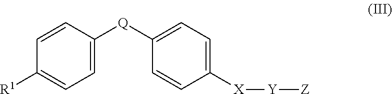

wherein
R¹ is halogen, OH, NH₂, OC₁₋₁₀alkyl unsubstituted or substituted by 1-3 halogen, or NH₂;

Q is absent or O;

X is O or S(O)₂;

Y is CH₂ or NH;

Z is C₅₋₁₄heteroaryl having 1 to 3 heteroatoms independently selected from N, O and S or

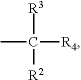

and
R², R³ and R⁴ are each independently H, C₁₋₁₀alkyl,

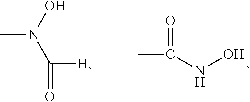

or unsubstituted or substituted C₅₋₁₄heteroaryl having 1 to 3 heteroatoms independently selected from N, O and S; or R² and R⁴ are taken together with the carbon atom to which each is attached form a 5 membered saturated heterocyclyl ring which is unsubstituted or substituted by CN or C₁₋₁₀alkyl, C₁₋₁₀alkylC₅₋₁₅aryl.

Preferably, when Q is absent, R¹ is OC(halogen)₃, X is O, Y is CH₂, Z is

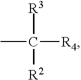

and R², R³ and R⁴ are each independently H,

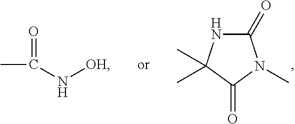

or R² and R⁴ are taken together with the carbon or nitrogen atom to form

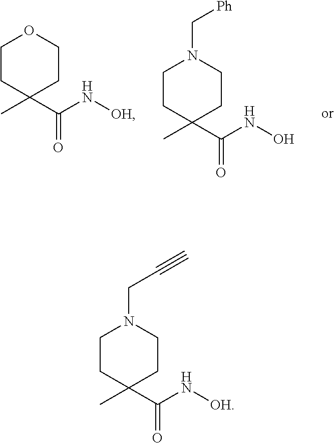 [SMILES: CC1(C(=O)NO)CCOCC1 ; CC1(C(=O)NO)CCN(Cc2ccccc2)CC1 ; C#CCN1CCC(C)(C(=O)NO)CC1]

Preferably, when Q is O; R¹ is halogen or OC(halogen)₃, X is S(O)₂, and Z is

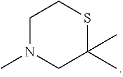

Preferably, when Q is O; R¹ is halogen or OC(halogen)₃, X is S(O)₂, Y is NH; Z is

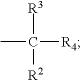

and R², R³ and R⁴ are each independently H, C₁₋₁₀alkyl,

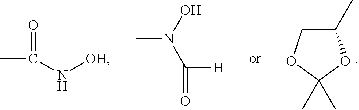

More preferably, the compound of Formula (III) is selected from the group consisting of:

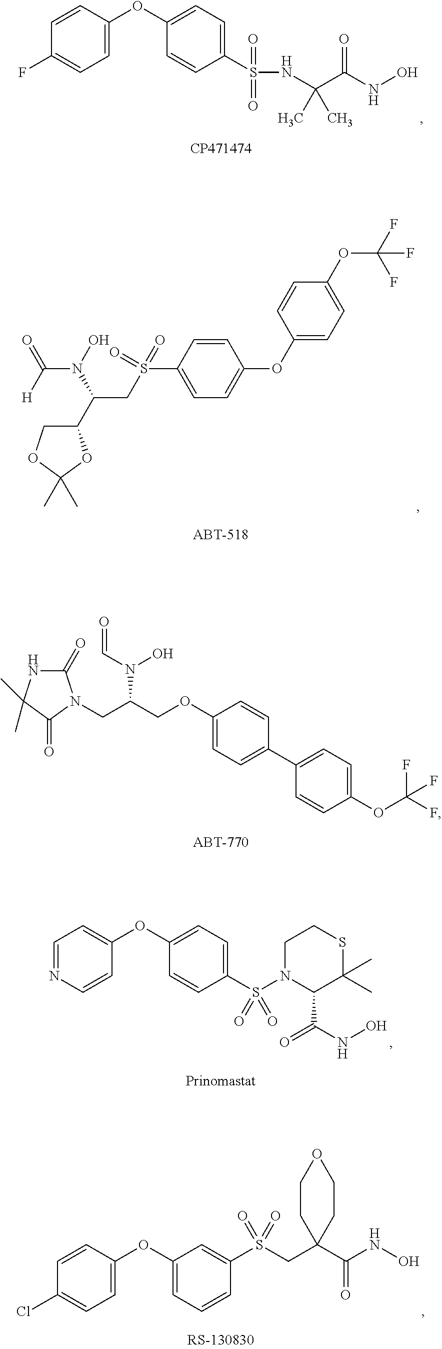 [SMILES: CC(C)(NS(=O)(=O)c1ccc(Oc2ccc(F)cc2)cc1)C(=O)NO ; CC1(C)OC[C@H]([C@@H](CS(=O)(=O)c2ccc(Oc3ccc(OC(F)(F)F)cc3)cc2)N(O)C=O)O1 ; CC1(C)NC(=O)N(C[C@@H](COc2ccc(-c3ccc(OC(F)(F)F)cc3)cc2)N(O)C=O)C1=O ; CC1(C)SCCN(S(=O)(=O)c2ccc(Oc3ccncc3)cc2)[C@H]1C(=O)NO ; O=C(NO)C1(CS(=O)(=O)c2cccc(Oc3ccc(Cl)cc3)c2)CCOCC1]

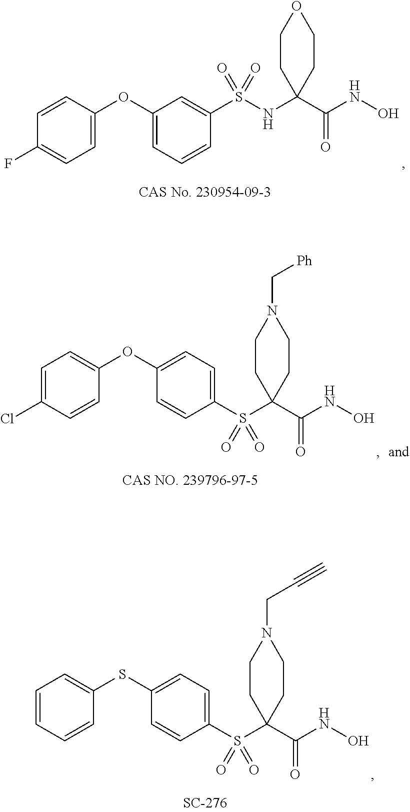 [SMILES: O=C(NO)C1(NS(=O)(=O)c2cccc(Oc3ccc(F)cc3)c2)CCOCC1 ; O=C(NO)C1(S(=O)(=O)c2ccc(Oc3ccc(Cl)cc3)cc2)CCN(Cc2ccccc2)CC1 ; C#CCN1CCC(C(=O)NO)(S(=O)(=O)c2ccc(Sc3ccccc3)cc2)CC1]

or a pharmaceutically acceptable salt, prodrug, solvate, stereoisomer or enantiomer thereof.

According to a further another embodiment of the disclosure, the MMP inhibitor is a compound having the following formula:

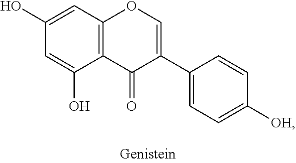 [SMILES: O=c1c(-c2ccc(O)cc2)coc2cc(O)cc(O)c12]

or a pharmaceutically acceptable salt, prodrug, solvate, stereoisomer or enantiomer thereof.

More preferably, the MMP inhibitor is Marimastat, Batimastat, CL-82198, Minocycline, Tetracycline or Doxycycline.

TGF-Beta Inhibitors for Use in a Method for Affecting the Expression of Lumican and/or Collagen Fibrillogenesis, and/or Treating Myopia and/or Keratoconus Disease

In an another embodiment, the disclosure provides a method for treating a disease medicated by expression of lumican and/or collagen fibrillogenesis, and/or treating myopia and/or keratoconus disease, comprising administering to the subject a therapeutically effective amount of a TGF-beta inhibitor.

Transforming growth factor-beta (TGF-beta) belongs to a large super-family of multifunctional polypeptide factors. TGF-beta is a potent inducer of growth arrest in many cell types, including epithelial cells. This activity is the basis of the tumor suppressor role of the TGF-beta signaling system in carcinomas. Other activities, including TGF-beta-induced epithelial-to-mesenchymal differentiation, contribute to cancer progression. PCT patent application WO 02/0948332 describes a genus of dihydropyrrolopyrazole compounds useful for treating disorders associated with enhanced TGF-beta signaling activity or overproduction. U.S. Pat. No. 7,638,537 and U.S. Pat. No. 7,635,702 provide pyrazole compounds and imidazole compounds as potent inhibitors of TGF-signaling pathway.

According to one embodiment of the disclosure, the TGF-beta inhibitor is selected from the group consisting of:

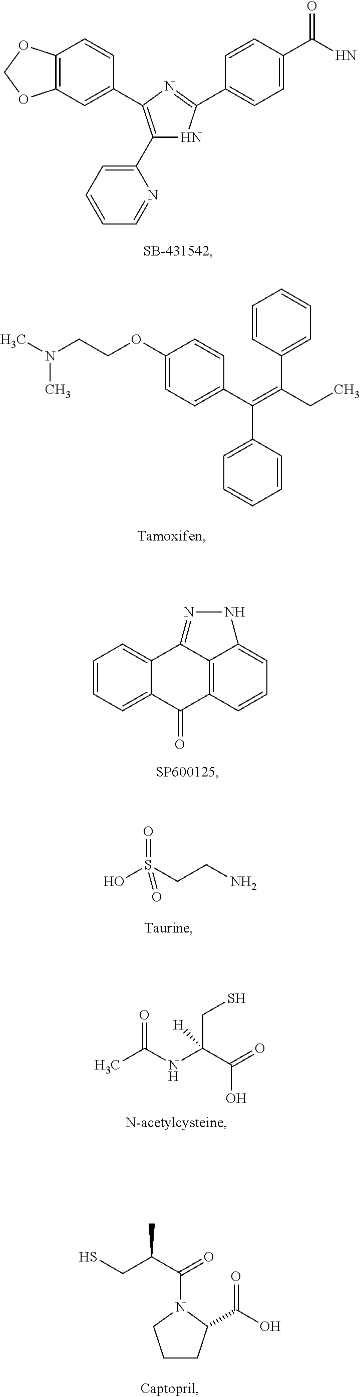

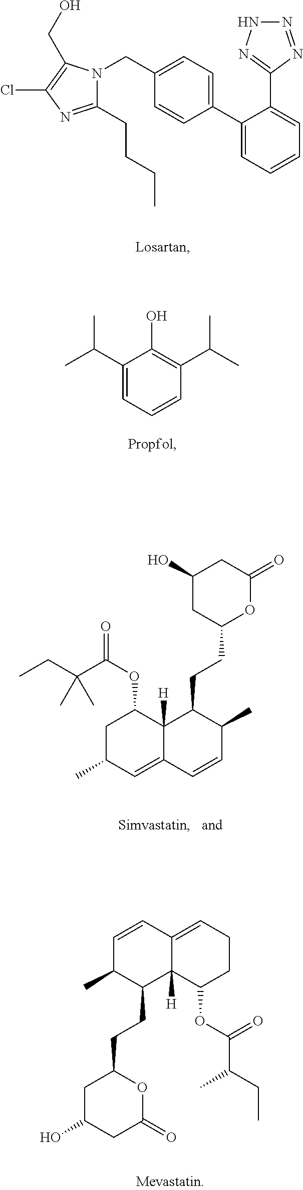

or a pharmaceutically acceptable salt, prodrug, solvate, stereoisomer or enantiomer thereof.

Preferably, the TGF-beta inhibitor is Losartan, N-acetylcysteine, Propofol and Captopril.

COX/LOX Inhibitors for Use in a Method for Affecting the Expression of Lumican and/or Collagen Fibrillogenesis, and/or Treating Myopia and/or Keratoconus Disease

In a further embodiment, the disclosure provides a method for treating a disease medicated by expression of lumican and/or collagen fibrillogenesis, and/or treating myopia and/or keratoconus disease, comprising administering to the subject a therapeutically effective amount of a COX/LOX inhibitor.

COX enzymes convert arachidonic acid to the prostaglandin endoperoxide PGH2, from which other prostaglandins are formed. A number of drugs inhibit the action of either the COX or the LOX enzymes.

According to the disclosure, the COX/LOX inhibitor is selected from the group consisting of:

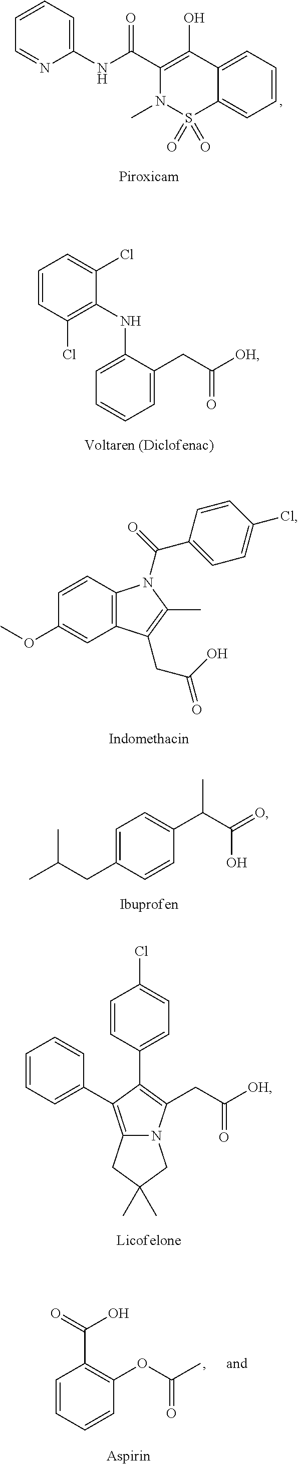 [SMILES: CN1C(C(=O)Nc2ccccn2)=C(O)c2ccccc2S1(=O)=O ; O=C(O)Cc1ccccc1Nc1c(Cl)cccc1Cl ; COc1ccc2c(c1)c(CC(=O)O)c(C)n2C(=O)c1ccc(Cl)cc1 ; CC(C)Cc1ccc(C(C)C(=O)O)cc1 ; CC1(C)Cc2c(-c3ccccc3)c(-c3ccc(Cl)cc3)c(CC(=O)O)n2C1 ; CC(=O)Oc1ccccc1C(=O)O]

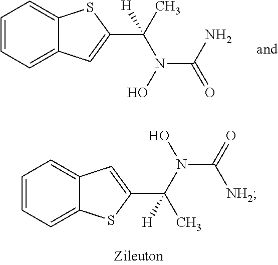 [SMILES: C[C@H](c1cc2ccccc2s1)N(O)C(N)=O ; C[C@@H](c1cc2ccccc2s1)N(O)C(N)=O]

or a pharmaceutically acceptable salt, prodrug, solvate, stereoisomer or enantiomer thereof.

Preferably, the COX/LOX inhibitor is Aspirin.

Anticholinergic & Muscarinic Serier Compounds for Use in a Method for Affecting the Expression of Lumican and/or Collagen Fibrillogenesis, and/or Treating Myopia and/or Keratoconus Disease

In a further embodiment, the disclosure provides a method for treating a disease medicated by expression of lumican and/or collagen fibrillogenesis, and/or treating myopia and/or keratoconus disease, comprising administering to the subject a therapeutically effective amount of an anticholinergic or muscarinic compound.

According to the disclosure, the anticholinergic or muscarinic compound is selected from the group consisting of:

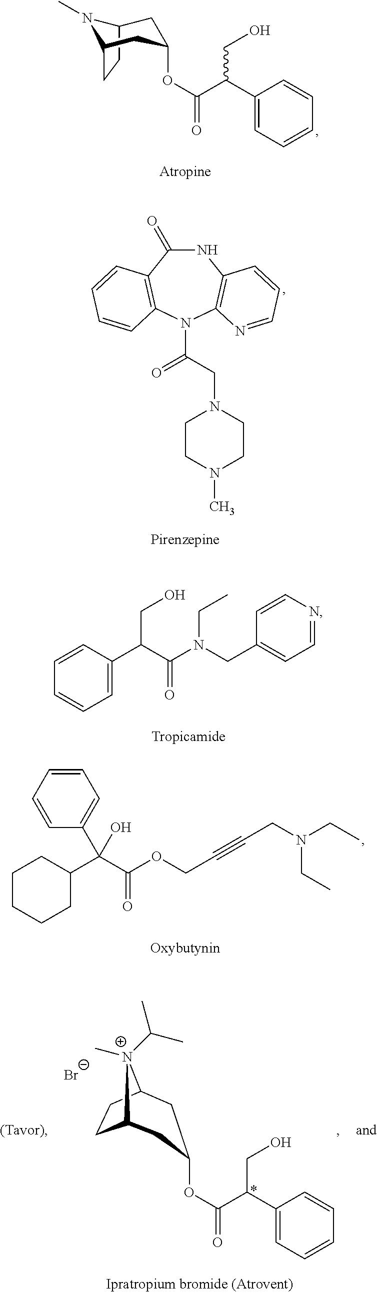

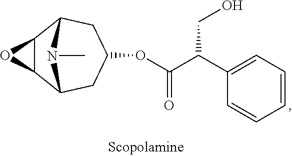

or a pharmaceutically acceptable salt, prodrug, solvate, stereoisomer or enantiomer thereof.

Preferably, the anticholinergic or muscarinic compound is atropine.

Illustrating examples of the candidate compounds are listed in the table below:

| Name | Type |
|---|---|
| Propofol | TGF beta activation |
| Simvastatin | TGF beta activation |
| Mevastatin | TGF beta activation |
| SB-431542 (4-[4-(1,3-benzodioxol-5- | TGF-beta receptor inhibitor |
| yl)-5-(2-pyridinyl)-1H-imidazol-2- | |
| yl]benzamide) | |
| Tamoxifen | TGF-Beta1 inhibitor |
| SB-505124 (2-[4-(1,3-Benzodioxol-5- | TGF-beta receptor inhibitor |
| yl)-2-(1,1-dimethylethyl)-1H-imidazol- | |
| 5-yl]-6-methyl-pyridine) | |
| RepSox (SB-4696) | TGF-beta receptor inhibitor |
| Captopril | TGFB inhibitor |
| SP600125[Anthrapyrazolone] | TGFB inhibitor |
| N-Acetylcysteine | Collagenase inhibitor |
| Vita min E succinate | TGF beta activation |
| Concanavalin A (Con A) | TGF beta activation |
| Statin | TGF beta activation |
| SB525334 (6-[2-tert-Butyl-5-(6- | TGF-beta receptor inhibitor |
| methyl-pyridin-2-yl)-1H-imidazol-4-yl]- | |
| quinoxaline) | |
| Doxorubicin | TGFB inhibitor |
| Norrin | TGFB inhibitor |
| AP 12009 (Trabedersen) | TGFB inhibitor |
| Doxycycline | MMP inhibitor |
| Genistein | MMP inhibitor |
| Marimastat | MMP inhibitor |
| Minocycline hydrochloride | MMP inhibitor |
| CL-82198 (N-[4-(4-Morpholinyl)butyl]- | MMP inhibitor |
| 2-benzofurancarboxamide | |
| hydrochloride) | |
| Ilomastat (GM6001) | MMP inhibitor |
| Batimastat | MMP inhibitor |
| CP471474 (2-[[[4-(4- | MMP inhibitor |
| Fluorophenoxy)phenyl]sulfonyl]amino]- | |
| N-hydroxy-2-methylpropanamide) | |
| Tetracycline | MMP inhibitor |
| Aspirin | COX inhibitor |
| naproxen | COX inhibitor |
| Indomethacin | COX inhibitor |
| Piroxicam | COX inhibitor |
| Zileuton | COX inhibitor |
| Voltaren | COX inhibitor |
| Iduprofen | COX inhibitor |
| Atropine | Anticholinergic |
| Tropicamide | Anticholinergic |
| Ipratropium bromide | Anticholinergic |
| Oxybutynin | Antimuscarinic |
| Scopolamine hydrobromide | Antimuscarinic |
| Pirenzepine dihydrochloride | Antimuscarinic |
| Taurine | Glycine receptors inhibitor |
| Losartan | Angiotensin-II receptor inhibitor |
| PD-166866 (6-aryl-pyrido[2,3- | FGF inhibitor |
| d]pyrimidines) | |
| PD-161570 (N-[6-(2,6-Dichlorophenyl)- | FGF inhibitor |
| 2-[[4- | |
| (diethylamino)butyl]amino]pyrido[2,3- | |
| d]pyrimidin-7-yl]-N′-(1,1- | |
| dimethylethyl)urea) | |
| PD 173074 (N-[2-[[4- | FGF inhibitor |
| (Diethylamino)butyl]amino]-6-(3,5- | |
| dimethoxyphenyl)pyrido[2,3- | |
| d]pyrimidin-7-yl]-N′-(1,1- | |
| dimethylethyl)urea) | |
| (E/Z)-BCI hydrochloride | FGF inhibitor |
| 1-Methyl-2-piperidinemethanol | FGF inhibitor |
| 2-Thiohydantoin | FGF inhibitor |
| SU5402 (2-[(1,2-Dihydro-2-oxo-3H- | FGF inhibitor |
| indol-3-ylidene)methyl]-4-methyl-1H- | |
| pyrrole-3-propanoic acid) | |
| PD166285 (the 6-aryl-pyrido[2,3- | FGF inhibitor |
| d]pyrimidines) | |
| N-Methyl-4-piperidinol | FGF inhibitor |
| PD-089828 (6-aryl-pyrido-[2,3- | FGF inhibitor |
| d]pyrimidines) | |
| NP603 ((Z)-3-(5-(6-(3,5- | FGF inhibitor |
| Dimethoxyphenyl)-2-oxo-1,2-dihydro- | |
| indol-3-ylidenemethyl)-2,4-dimethyl- | |
| 1H-pyrrol-3-yl)-propionic acid) | |
| SU4984 ([3-[4-(1-formylpiperazin-4- | FGF inhibitor |
| yl)-benzylidenyl]-2-indolinone] | |
| TSU-68 (SU 6668) (Orantinib) | FGF inhibitor |
| Brivanib | FGF inhibitor |
| BIBF1120 (Vargatef) | FGF inhibitor |
| Ponatinib (AP24534) | FGF inhibitor |
| Danusertib (PHA-739358) | FGF inhibitor |
| Masitinib | FGF inhibitor |
| Brivanib (BMS-540215) | FGF inhibitor |
| Brivanib alaninate (BMS-582664) | FGF inhibitor |

Preferred examples include, but not limited to, those listed in the table below.

Name

Atropine
Tropicamide
Ipratropium bromide (Atrovent)
Oxybutynin (Tavor)
Scopolamine hydrobromide
Pirenzepine dihydrochloride
SB 431542 (4-[4-(1,3-benzodioxol-5-yl)-5-(2-pyridinyl)-1H-imidazol-2-
yl]benzamide)
Tamoxifen
SB-505124 (2-[4-(1,3-Benzodioxol-5-yl)-2-(1,1-dimethylethyl)-1H-
imidazol-5-yl]-6-methyl-pyridine)
RepSox (SB-4696)
Doxycycline hyclate (Dermostat, Periostat)
Genistein
Marimastat
Taurine
Minocycline hydrochloride
n-acetylcysteine
Aspirin
Propofol
SP600125 (Anthrapyrazolone)
Zileuton
Mevastatin
Indomethacin
Piroxicam
Captopril
Simvastatin

Any of the above-mentioned compounds can be combined with a pharmaceutically acceptable carrier to form a formulation, composition, combination or preparation (each term can be used interchangeable). The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present disclosure within or to the subject such that it can perform its intended function. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredients which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluents.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this disclosure. Such solutions are useful for the treatment of any ophthalmic disease.

Pharmaceutical compositions of this disclosure suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The preparations of the present disclosure may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous injection administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, epidural, intraorbital, intracardiac, intradermal, intraperitoneal, transtracheal, subcutaneous, subcuticular, intraarticular, subcapsular, subarachnoid, intraspinal and intrasternal injection and infusion.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present disclosure, which may be used in a suitable hydrated form, and/or the pharmaceutical compositions of the present disclosure, are formulated into pharmaceutically acceptable dosage forms by conventional methods known to those of ordinary skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

The disclosure proposes that Lumican, one of several SLRPs, plays an important role in the regulation of fibrillogenesis or the genes affecting the size of eyeballs in zebrafish, in addition to playing an important role in clinical myopia The disclosure is to use our established zebrafish model to further identify the drugs affecting the expression of lumican and collagen fibrillogenesis and the regulation of eyeball size. Test compounds are tested based on the regulation of lumican and collagen synthesis through TGF-β pathway and subsequent MMP2 and TIMP regulation, test compounds are tested. In the disclosure, about 30 clinically-available and FDA-approval drugs which are currently used in clinical and relevant regulation of the TGF-β pathway or MMP and TIMP activity were tested. The results revealed that MMP inhibitors (marinastat, doxycycline and minocycline), collagenase inhibitors (n-acetylcysteine), TGF-β pathway activators (propofol), TGF-β inhibitor (Captopril) and Cox inhibitor (Aspirin) are effective candidate compounds.

### EXAMPLES

### Materials and Methods

### Aquaculture

Zebrafish are raised and maintained according to previously established protocols (Soules K A, Link B A. Morphogenesis of the anterior segment in the zebrafish eye. BMC Dev Biol 2005; 5:12). All experiments are performed on Tuebingen AB zebrafish raised at 28° C. on a 14-h light and 10-h dark cycle and maintained using standard methods. Embryos are staged according to morphological criteria (somite number) (Kimmel C B, Ballard W W, Kimmel S R, et al. Stages of embryonic development of the zebrafish. Dev Dyn 1995; 203(3):253-310) and timed in hours after fertilization. Embryos are generated by natural pair-wise mating, as described in the zebrafish handbook (Westerfield M. The Zebrafish Book; A Guide for the Laboratory Use of Zebrafish (Brachydanio rerio). University of Oregon Press, Eugene, 2nd edition 300P., 1993). For each mating, 4±5 pairs are set up and, on an average; 100±150 embryos per pair are generated. The zebrafish embryo is optically transparent, making it possible to detect functional and morphological changes in internal organs without having to kill or dissect the organism. Chorions are removed manually with Dumont Watchmaker's Forceps No. 5.

### Zebrafish Lumican Clone

The zebrafish genome has now been sequenced by the Sanger Center, and there have been substantial annotations on the genome conducted by the trans-National Institutes of Health Zebrafish Genome Initiative. To identify the zebrafish expressed sequence tag (EST) clone encoding a putative protein that shares a high sequence similarity with the human and mouse SLRP family proteins, we applied a Basic Local Alignment Search Tool (BLAST) analysis of the GenBank database using the full-length human lumican cDNA sequence. An approximately 4.6 kb Not I/MluI zebrafish genomic DNA fragment containing the 5′ portion of the zebrafish Lumican gene is amplified by polymerase chain reaction (PCR) and subcloned into the pBluescript SK vector (Stratagene, La Jolla, Calif.). The insert is sequenced, using T3, T7 and walk-in primers, by the DNA core of the Department of Molecular Genetics at the National Taiwan University. The 5′- and 3′-ends of the zLum mRNA are amplified using the 5′-Rapid Amplification of cDNA END (5′-RACE) and 3′-RACE Systems, respectively (Invitrogen, Carlsband, Calif.). For the 5′-RACE experiment, 1 μg of total RNA from zebrafish eyes is reverse transcribed with a first lumican-specific primer corresponding to a sequence in exon 2 of the zLum gene. The RNA templates are degraded by treatment with an RNase mix. A poly-dCTP tail is added to the 3′-end of the cDNAs with terminal deoxynucleotidyl transferase. The cDNA is amplified with a second gene-specific primer corresponding to a sequence from the junction between exon 1 and exon 2 in conjunction with the abridged anchor primer. The resulting PCR products are diluted 100-fold and used as templates to be reamplified with a third gene-specific primer in conjunction with the universal amplification primer. For 3′-RACE, PCRs are performed using a forth gene-specific primer corresponding to a sequence in exon 3 of the zLum gene. The cycling conditions are: 34 cycles of 94° C. for 1 min, 55° C. for 1 min, and 72° C. for 3 min followed by a 10-min extension at 72° C. at the end of the cycles. Finally, the 5′-RACE and 3′-RACE PCR products are gel purified, and the sequences are determined with a dideoxy sequencing protocol. The transcription initiation and termination sites of the zLum gene are determined by a sequence comparison between genomic DNA, the 5′-RACE product, and the 3′-RACE product, respectively (Yeh L K, Liu C Y, Kao W W, et al. Knockdown of zebrafish lumican gene (zlum) causes scleral thinning and increased size of scleral coats. J Biol Chem 2010; 285(36):28141-55).

First lumican-specific primer:
5′-AGTAGAGGTATTTGATTCCGGTC-3′;
Second lumican-specific primer:
5′-GCACAAGAAGGTGATGAAACG-3′;
Third lumican-specific primer:
5′-CAGACTTAGAAGTCCAGCCAAC-3′;
Forth gene-specific primer:
5′-GCCTCAGAGATCATCTTTGAATAG-3′;
Abridged anchor primer:
5′-GGCCACGCGTCGACTAGTACGGGIIGGGIIGGGIIG-3′;
Universal amplification primer:
5′-CUACUACUACUAGGCCACGCGTCGACTAGTAC-3′.

### Morpholino Knockdown

Morpholinos are chemically modified antisense oligonucleotides that can be designed to hybridize to the translation-initiation or splicing acceptor/donor sites of specific mRNAs (Nasevicius A, Ekker S C. Effective targeted gene ‘knockdown’ in zebrafish. Nat Genet 2000; 26(2):216-20). A morpholino-antisense oligonucleotide (Gene Tools, Philomath, Oreg.) are designed and synthesized to target the 5′-untranslated and/or flanking regions, including the translation start codon of the respective genes. The MO sequence is designed as follows: zLum-MO, 5′-GATCCCAGAGCAAACATGGCTGCAC-3′. This oligonucleotide complemented the sequence from −8 through +17 with respect to the translation initiation codon. A random sequence MO (RS-MO) serves as a control for zLum-MO: 5′-CCTCTTACCTCAGTTACAATTTATA-3′. This RS-MO is obtained from Gene Tools as a standard control oligonucleotide with no target specificity (Yeh L K, Liu C Y, Kao W W, et al. Knockdown of zebrafish lumican gene (zlum) causes scleral thinning and increased size of scleral coats. J Biol Chem 2010; 285(36):28141-55).

Morpholino is resuspended in sterile water to a concentration of 1 mmol/L and diluted to 680 ng/nL with sterile water. The morpholinos are injected at the single-cell stage in a volume of 0.0023 nL. Here, we identified the effects of morpholinos on protein levels are assayed with western blotting from injected embryos with GAPDH as a control.

### Whole-Embryo In Situ Hybridization

The main advantage of whole-mount ISH is that it is a quick and efficient method to establish spatial and temporal gene expression patterns in embryos and early larvae. Embryos are obtained at different stages and fixed in 4% paraformaldehyde in 1×PBS overnight at 4° C. After rinsed with PBS 3 times, we transferred these embryos into 100% methanol, and stored at −20° C. until use. All embryos are treated with 0.003% phenylthiourea (PTU) to prevent melanogenesis. Whole mount RNA in situ hybridization is carried out according to the nature protocol (Thisse C, Thisse B. High-resolution in situ hybridization to whole-mount zebrafish embryos. Nat Protoc 2008; 3(1):59-69). The hybridization signals are visualized with anti-digoxigenin (DIG) antibody-alkaline phosphatase conjugates using procedures recommended by Roche (Roche Applied Science, Indianapolis, Ind.).

### Antibody

Zebrafish Lumican Antibody—An affinity-purified anti-zLum antibody against a synthetic peptide N-terminal peptide (N′-CNERNLKFIPIVPTGIKY-C′) corresponding to the 18 N-terminal amino acid residues deduced from the zLum cDNA is generated to detect zebrafish lumican. The peptides are conjugated to keyhole limpet hemocyanin for antibody production in rabbits. The antibodies are purified through an immune absorbent column of the above zebrafish Lumican oligopeptide conjugated to Sulfolink gel (Pierce, Rockford, Ill.) according to the manufacturer's instructions. Fractions containing purified anti-zebrafish lumican antibody are pooled and concentrated, and the protein concentration is measured by spectrophotometer at 280 nm (Yeh L K, Liu C Y, Kao W W, et al. Knockdown of zebrafish lumican gene (zlum) causes scleral thinning and increased size of scleral coats. J Biol Chem 2010; 285(36):28141-55).

We used several antibodies to evaluate the effects of lumican knockdown and all can be obtained from different commercial supplies. Rabbit anti-TGFβ2, Rabbit anti-MMP-2, Goat anti-TIMP-2, Goat anti-Col1a1 (L-19) and Goat anti-PDI are obtained from Santa Cruz. Mouse anti-GAPDH is obtained from Abnova.

### Western Blotting

Human sclera cells will be seeded in 6-well culture plates at 4×10⁵ cells/well and incubated with different concentrations of MMP inhibitors at 37° C. for 24 hours. The culture without MMP inhibitors will act as controls. After 24 hours incubation, cells will be harvested for protein extraction. Proteins are extracted and homogenized in RIPA buffer containing protease inhibitor. Protein content is quantified by spectrophotometry. Samples with equal protein content are electrophoresed on 10% polyacrylamide gels and electrophoretically transferred to PVDF membranes. The blot membranes are incubated with PBS solution containing 5% skim milk overnight at 4° C. to block nonspecific antigens and then incubated with primary antibody diluted for 1-2 hours. Primary antibody used in this experiment are as follows: anti-TGFβ1, anti-TGFβ2, anti-TGFβ3, anti-MMP2, anti-MMP9, anti-TIMP2 and GAPDH. After the primary antibody reaction, the membranes will be incubated with horseradish peroxidase-conjugated goat anti-mouse IgG or goat anti-rabbit IgG as the secondary antibody at room temperature for 1 h, detected by Chemiluminescence Reagent Plus, and exposed to film. We will compare the protein expression pattern between cell treated with/without different MMP inhibitors.

zLumican Promoter Transgenic Fish

Gene function can be rapidly and robustly studied in zebrafish using antisense morpholino oligonucleotides (Nasevicius A, Ekker S C. Effective targeted gene ‘knockdown’ in zebrafish. Nat Genet 2000; 26(2):216-20; Heasman J. Morpholino oligos: making sense of antisense? Dev Biol 2002; 243(2):209-14). Furthermore, techniques for generating transgenic lines 70 (Davidson A E, Balciunas D, Mohn D, et al. Efficient gene delivery and gene expression in zebrafish using the Sleeping Beauty transposon. Dev Biol 2003; 263(2):191-202; Kurita K, Burgess S M, Sakai N. Transgenic zebrafish produced by retroviral infection of in vitro-cultured sperm. Proc Natl Acad Sci USA 2004; 101(5):1263-7), targeted mutations (reverse genetics) (Wienholds E, Schulte-Merker S, Walderich B, Plasterk R H. Target-selected inactivation of the zebrafish rag1 gene. Science 2002; 297(5578):99-102) and cloning by nuclear transfer (Lee K Y, Huang H, Ju B, et al. Cloned zebrafish by nuclear transfer from long-term-cultured cells. Nat Biotechnol 2002; 20(8):795-9) have been developed. Here, we established a transgenic line expressed under the control of the zebrafish lumican promoter. Genomic DNA, both 1.7 kb and 0.48 kb from the 5′-untranslated region of the zLum gene, are amplified with specific PCR primers and inserted into the multiple cloning sites of pBluescript II SK vectors (Stratagene, La Jolla, Calif.) containing an EGFP sequence 59. The recombinant plasmids are prepared in Escherichia coli DH5α and purified with a QIAGEN Plasmid Purification Maxi kit. Purified plasmid DNA is adjusted to 50 ng/μl in distilled water and microinjected into one-cell-stage zebrafish embryos under a dissecting microscope. The following day, embryos with GFP expression are imaged and selected by using a Leica dissection scope equipped with epifluorescence (MZFLII). Only embryos displaying fluorescence are grown to adulthood. Pairs of sibling adults grown from injected embryos with fluorescence are intercrossed to identify germ line founders. Subsequently, individual adults from positive pairs are outcrossed to identify the individual founder fish. These functional and morphological changes may be further highlighted by this lumican promoter transgenic fish.

### PCR Primers:

Forward primer I:
5′-ATAAGAATGCGGCCGCTCCATTAATTCGACAGACCAG-3′;
Forward primer II:
5′-ATAAGAATGCGGCCGCAGGTAGACAACACGGTTATGT-3′;
Reverse primer:
5′-CGACGCGTGGCTGCACAACTTAAATTAAACCT-3′;

### Chemicals Used for the Primary Drug Screening

The chemicals and drugs for drug screening will include TGF-receptor inhibitors (Atropine [symbol] tropicamide [symbol] ipratropium bromide (Atrovent) [symbol] oxybutynin (Tavor) [symbol] scopolamine hydrobromide [symbol] Pirenzepine dihydrochloride [symbol] SB 431542 [symbol] Tamoxifen [symbol] SB-505124 [symbol] RepSox (SB-4696) [symbol] Doxycycline hyclate (Dermostat, Periostat) [symbol] Genistein [symbol] Marimastat [symbol] Taurine [symbol] Minocycline hydrochloride [symbol] n-acetylcysteine [symbol] Losartan [symbol] aspirin [symbol] zileuton [symbol] SP600125 [symbol] Propofol [symbol] Statin [symbol] indomethacin [symbol] Ibuprofen [symbol] naproxen [symbol] piroxicam [symbol] nabumetone [symbol] Licofelone [symbol] Captopril [symbol] Procyanidin [symbol] Heterotaxin [symbol] Simvastatin [symbol] Lovastatin [symbol] Rosuvastatin). All of these compounds have been well investigated before for their pharmacological activities against the presumed pathways involved the lumican-regulated collagen fibrillogenesis.

### Results

We have successfully established a zebrafish model to study ocular development and diseases and characterized lumican gene (zLum) expression in the cornea and the sclera of zebrafish. Knockdown of zLum causes scleral thinning and increased size of scleral coats during the ocular development of zebrafish, compatible with the clinical findings in child myopia. As shown in FIGS. 4 and 5 of The Journal of Biological Chemistry, 2010, Vol. 285, No. 36, pp. 28141-28155, it is clearly demonstrated the expression of zebrafish lumican (zLum) in the zebrafish, especially in the scleral coat, cornea and periocular matrices. Interestingly, the lumican in the scleral coat is non-sulfated in contrast with that of sulfated lumican in the corneal stroma.

After lumican underwent knockdown, in FIGS. 1 to 5, the scleral coat became enlarged and was similar to the scleral changes of human myopia, i.e. axial elongation. As mentioned above, we also showed the alteration of a SNP in the lumican promoter and its haplotype were strongly associated with development of high myopia in Taiwanese population. Our animal study recaptured these findings in the human myopia in which the importance of lumican gene in the development of axial elongation was emphasized.

In FIG. 6, we showed that the phenotype of lumican knockdown in zebrafish could be rescued with the TGF-β. The lumican knockdown fish also demonstrated by the increased expression of MMP2 and decreased expression of TIMP which further confirmed the role of lumican in the regulation of scleral remodeling as shown in experimental myopia of other species. Importantly, the scleral coat enlargement could be inhibited with the administration of atropine in lumican knockdown fish (FIGS. 7 to 9). The expression of MMP-2 and TIMP also returned to the normal level with this treatment.

We have tested about 30 clinically-available drugs relevant to TGF-β pathway. The first drug, marinastat (BB 2516), a proposed anti-neoplastic drug, acting as a broad-spectrum matrix metalloproteinase inhibitor, is also considered a good candidate. Our preliminary results revealed that marinastat could prevent the scleral coat enlargement very efficiently in zLumMO knockdown fish (2% of scleral enlargement in experimental group vs. 30% of scleral enlargement in control group). The results of marinastat indeed showed the MMPs are the effectors and targets for scleral coat enlargement after lumican knockdown. Marinastat can be a potential target for myopia prevention and clinical drug testing.

Tetracyclines have been used both systemically and locally in the treatment of various infections caused by gram-negative bacteria. During recent years it has been established that tetracyclines exert biological functions entirely independent of their antimicrobial property. Furthermore, several investigations involving both in vitro and in vivo animal studies have shown that tetracycline antibiotics and their chemically modified analogues with no antimicrobial activity can inhibit mammalian collagenase activity and collagen breakdown. Doxycycline and minocycline were second-generation tetracyclines. Doxycycline and chemically modified tetracyclines CMT-1 and CMT-6 had direct inhibitory effects on both 92-kDa (MMP-9) and 72-kDa (MMP-2) gelatinaes. Minocycline also inhibit various MMPs, including MMP-9 and MMP-2. These drugs were considered as a good candidate. Our results revealed that doxycycline and minocycline could prevent the scleral coat enlargement very efficiently in zLum-morpholinos (MO) knockdown model (6.8% and 4.7% of scleral enlargement in experimental group vs. 30% of scleral enlargement in control group). The another antibiotics, minocycline, also presented the effectiveness on the prevention of sclera enlargement. Minocycline belongs to the second generation class of cyclines. Minocycline has an anti-infectious property with a spectrum similar to that of other cyclines, notably against Chlamydias, Treonema and Proprionibacterium acenes. The anti-inflammatory and anti-collagenase activity associated with this anti-infectious action is greater than that of first generation cyclines specifically with a modulator effect on epidermal cytokines. Therefore, it is reasonable to expect tetracycline to demonstrate efficacy in the collagen synthesis of the scleral coat. Aspirin causes several different effects in the body, mainly the reduction of inflammation, analgesia (relief of pain), the prevention of clotting, and the reduction of fever. Aspirin's ability to suppress the production of prostaglandins and thromboxanes is due to its irreversible inactivation of the cyclooxygenase (COX) enzyme. Cyclooxygenase is required for prostaglandin and thromboxane synthesis. Aspirin acts as an acetylating agent where an acetyl group is covalently attached to a serine residue in the active site of the COX enzyme. This makes aspirin different from other NSAIDs (such as diclofenac and ibuprofen), which are reversible inhibitors. Our results revealed that aspirin could prevent the scleral coat enlargement very efficiently in zLum-MO knockdown model (9.6% of scleral enlargement in experimental group vs. 30% of scleral enlargement in control group).

N-acetylcysteine, an effective antioxidant which inhibit the formation of extracellular reactive oxygen intermediates 128, also was a collagenase inhibitor. Several papers reported that N-acetylcysteine shows inhibition of matrix MMP-2 expression and activity. Our results revealed that n-acetylcysteine could prevent the scleral coat enlargement very efficiently in zLum-morpholinos (MO) knockdown model (11.7% of scleral enlargement in experimental group vs. 30% of scleral enlargement in control group).

Propofol (2,6-diisopropylphenol) is one of the most popular agents used to induce anesthesia in surgical procedures for long-term sedation and to treat postoperative nausea in critically-ill patients. Propofol could induce endothelial cells to express latent TGF-β, which was converted into active TGF-β by PBMCs in vivo. Our results revealed that propofol also could prevent the scleral coat enlargement very efficiently in zLum-MO knockdown model (12% of scleral enlargement in experimental group vs. 30% of scleral enlargement in control group).

In summary, the results of marinastat, doxycycline, minocycline, n-acetylcysteine, aspirin, Propofol showed the TGF-β and MMPs are the effectors and targets for scleral coat enlargement after lumican knocking down. These drugs could be a potential target for myopia prevention and clinical drug testing. Accordingly, we have proven zebrafish was an excellent in vivo animal model to observe the development of axial myopia and for screening compounds in treating myopia. The big eye ratios of the zebrafish treated with marimastat, doxycycline, captopril, minocycline hydrochloride, atropine, aspirin, propofol and N-acetylcysteine are shown in FIG. 10. FIG. 11 (a)-(e) shows FIG. 11 shows the big eye ratios of the zebrafish treated with tetracycline, minocycline, doxycycline, marimastat and batimastat at various concentrations. Other test compounds and their big eye ratios are shown in the table below.

| | | Conc. I | | Big eye |
|---|---|---|---|---|
| | Name | choice | | rate(%) |
| 1 | Atropine | 0.50% | | 14.5% |
| 2 | Tropicamide | 1 | mM | 22.3% |
| 3 | Ipratropium bromide | 50 | mM | 26.0% |
| | (Atrovent) | | | |
| 4 | Oxybutynin (Tavor) | 2 | uM | 22.7% |
| 5 | Scopolamine hydrobromide | 100 | uM | 27.0% |
| 6 | Pirenzepine dihydrochloride | 0.25% | | 19.1% |
| 7 | SB 431542 | 25 | uM | 23.4% |
| 8 | Tamoxifen | 20 | uM | 17.7% |
| 9 | Doxycycline hyclate (Dermostat, | 100 | ppm | 6.8% |
| | Periostat) | | | |
| 10 | Genistein | 25 | uM | 36.2% |
| 11 | Marimastat | 50 | uM | 2% |
| 12 | Taurine | 0.2M | | 21.8% |
| 13 | Minocycline hydrochloride | 50 | uM | 4.7% |
| 14 | n-acetylcysteine | 10 | uM | 11.7% |
| 15 | Aspirin | 50 | mg/L | 9.6% |
| 16 | Propofol | 0.5 | mM | 12.0% |
| 17 | SP600125 | 2.5 | uM | 29.5% |
| 18 | Zileuton | 100 | uM | 21.3% |
| 19 | Mevastatin | 0.01 | nM | 27.0% |
| 20 | Indomethacin | 10 | uM | 18% |
| 21 | Piroxicam | 10 | uM | 22% |
| 22 | Captopril | 1 | mM | 7.6% |
| 23 | Simvastatin | 0.1 | uM | 26.0% |

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 CFR 1.77 or otherwise to provide organizational cues. These headings shall not limit or characterize the embodiment(s) set out in any claims that may issue from this disclosure. Specifically and by way of example, although the headings refer to a “Technical Field,” the claims should not be limited by the language chosen under this heading to describe the so-called field. Further, a description of a technology in the “Background” is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

What is claimed is:

1. A method for treating a disease mediated by expression of lumican and/or collagen fibrillogenesis, and/or treating myopia and/or keratoconus disease, comprising administering to the subject a therapeutically effective amount of a MMP inhibitor.

2. The method of claim 1, wherein the MMP inhibitor is a peptidomimetic hydroxamate MMP inhibitor having the following Formula (I) or a pharmaceutically acceptable salt, prodrug, solvate, stereoisomer or enantiomer thereof, 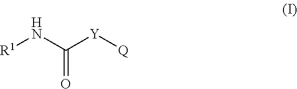
wherein
Q is absent or 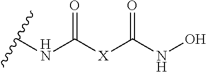
X is C₁₋₁₀ alkylene, C₂₋₁₀ alkenylene or C₂₋₁₀ alkynylene, unsubstituted or substituted by one or more OH, C₁₋₁₀ straight or branched alkyl, C₂₋₁₀ straight or branched alkenyl, C₁₋₁₀alkylC₅₋₁₅aryl, C₁₋₁₀alkenylC₅₋₁₅aryl, C₁₋₁₀alkynylC₅₋₁₅aryl, C₁₋₁₀alkylsulfanylC₅₋₁₅aryl, C₁₋₁₀alkylsulfonylC₅₋₁₅aryl, C₁₋₁₀alkylsulfinylC₅₋₁₅aryl, C₁₋₁₀alkyloxy or C₅₋₁₅aryl;
Y is C₁₋₁₀ alkylene, C₂₋₁₀ alkenylene or C₂₋₁₀ alkynylene, unsubstituted or substituted by one or more OH, C₁₋₁₀ straight or branched alkyl, C₂₋₁₀ straight or branched alkenyl, C₁₋₁₀alkylC₅₋₁₅aryl, C₁₋₁₀alkenylC₅₋₁₅aryl, C₁₋₁₀alkynylC₅₋₁₅aryl, C₁₋₁₀alkylsulfanylC₅₋₁₅aryl, C₁₋₁₀alkylsulfonylC₅₋₁₅aryl, C₁₋₁₀alkylsulfinylC₅₋₁₅aryl, C₁₋₁₀alkyloxy, C₅₋₁₅aryl, C₁₋₁₀alkylC₅₋₁₅aryl, C₅₋₁₄heteroaryl, C₁₋₁₀alkylC₅₋₁₅heteroaryl, or C₁₋₁₀alkylsulfanylC₅₋₁₅heteroaryl, provided that when Q is absent, Y is C₅₋₁₄heteroaryl; wherein the heteroaryl is optionally substituted and has 1 to 3 heteroatoms independently selected from N, O and S; and
R¹ is H, OH, C₁₋₁₀alkyl, C₂₋₁₀alkenyl, C₂₋₁₀alkynyl, C₅₋₁₅aryl, C₁₋₁₀alkylC₅₋₁₅aryl, C₅₋₁₄heteroaryl, or C₁₋₁₀alkylC₅₋₁₄heteroaryl.

3. The method of claim 2, wherein when
Q is 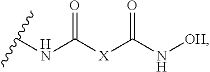
X is —CH₂—, —CH(CH₂CH₂(CH₃)₂)—, or —CH₂CH₂—, —CH(CH₂CH₂(CH₃)₂)CH(CH₃)—, —CH(CH₂CH₂(CH₃)₂)CH(CH₂—S-phenyl)-, —CH(CH₂CH₂(CH₃)₂)CH(OCH₃)—, —CH(CH₂CH₂(CH₃)₂)—, or —CH₂CH₂—, —CH(CH₂CH₂(CH₃)₂)CH(CH₃)—, —CH(CH₂CH₂(CH₃)₂)CH₂—, —CH(CH₂CH₂(CH₃)₂)CH(OH)—, or —CH(CH₂CH₂(CH₃)₂)CH(CH₂—S-thienyl)-;
Y is —CH(CH₂-phenyl)-, —CH(C(CH₃)₃)— or —CH(CH₂-indolyl)-; and
R₁ is CH₃ or phenyl.

4. The method of claim 2, when Q is absent,
(a) Y is 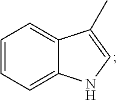
or
(b) Y is 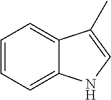
and
R₁ is C₅₋₁₅ heteroaryl; or
(c) Y is 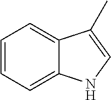
and

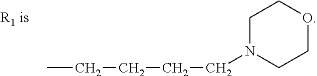

5. The method of claim 2, wherein the compound of Formula (I) is selected from the group consisting of: 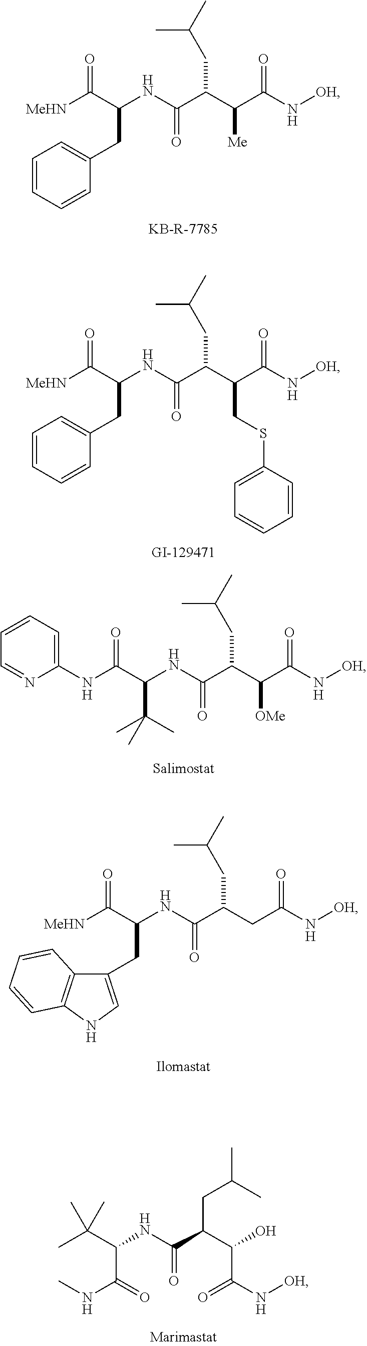 [SMILES: CNC(=O)[C@H](Cc1ccccc1)NC(=O)[C@H](CC(C)C)[C@H](C)C(=O)NO ; CNC(=O)[C@H](Cc1ccccc1)NC(=O)[C@H](CC(C)C)[C@H](CSc1ccccc1)C(=O)NO ; CO[C@H](C(=O)NO)[C@@H](CC(C)C)C(=O)N[C@H](C(=O)Nc1ccccn1)C(C)(C)C ; CNC(=O)[C@H](Cc1c[nH]c2ccccc12)NC(=O)[C@@H](CC(=O)NO)CC(C)C ; CNC(=O)[C@@H](NC(=O)[C@@H](CC(C)C)[C@H](O)C(=O)NO)C(C)(C)C] 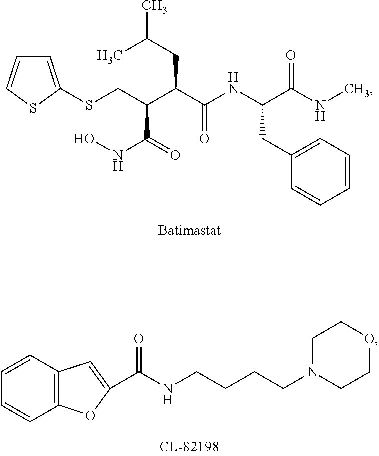 [SMILES: CNC(=O)[C@H](Cc1ccccc1)NC(=O)[C@H](CC(C)C)[C@H](CSc1cccs1)C(=O)NO ; O=C(NCCCCN1CCOCC1)c1cc2ccccc2o1]
a pharmaceutically acceptable salt thereof, a prodrug thereof, a solvate thereof, a stereoisomer thereof, an enantiomer thereof, and combinations thereof.

6. The method of claim 5, wherein the compound is CL-82198, Marimastat, or Batimastat.

7. The method of claim 1, wherein the MMP inhibitor is a tetracyclic-based MMP inhibitor having the following Formula (II) or a tautomer or pharmaceutically acceptable salt, prodrug or solvate thereof, 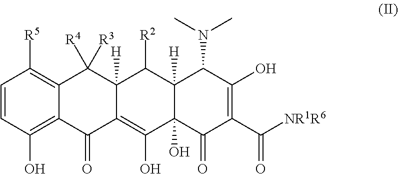
wherein
R¹ and R⁶ are each independently H, C₁₋₁₀alkylC₅₋₁₄heteroaryl, or C₁₋₁₀NR⁷R⁸;
R² is hydrogen or OH;
R³ and R⁴ are each independently H, OH, NH₂, NO, CN, C₁₋₁₀alkyl, C₁₋₁₀alkenyl or C₁₋₁₀alkynyl;
R⁵ is hydrogen, halogen, NH₂, OH, NO, CN, C₁₋₁₀ alkyl, NHC₁₋₁₀alkyl, N(C₁₋₁₀alkyl)₂, C₅₋₁₅aryl or C₅₋₁₄heteroaryl; and
R⁷ and R⁸ are each independently H, C₁₋₁₀alkyl C₁₋₁₀alkylNH₂COOH or taken together with the nitrogen atom to which each is attached form a 3 to 8 membered heteroaryl;
wherein heteroaryl has 1 to 3 heteroatoms independently selected from N, O and S.

8. The method of claim 7, wherein R¹ is H; R⁶ is H, —CH₂-pyrrolyl, —CH₂—NH—CH₂—CH₂—CH₂—CH₂—CH(NH2)-COOH; R² is H or oxo; R³ is H or OH; R⁴ is H or OH and R⁵ is NH₂, N(CH₃)₂ or halogen.

9. The method of claim 7, wherein the compound of Formula (II) is selected from the group consisting of: 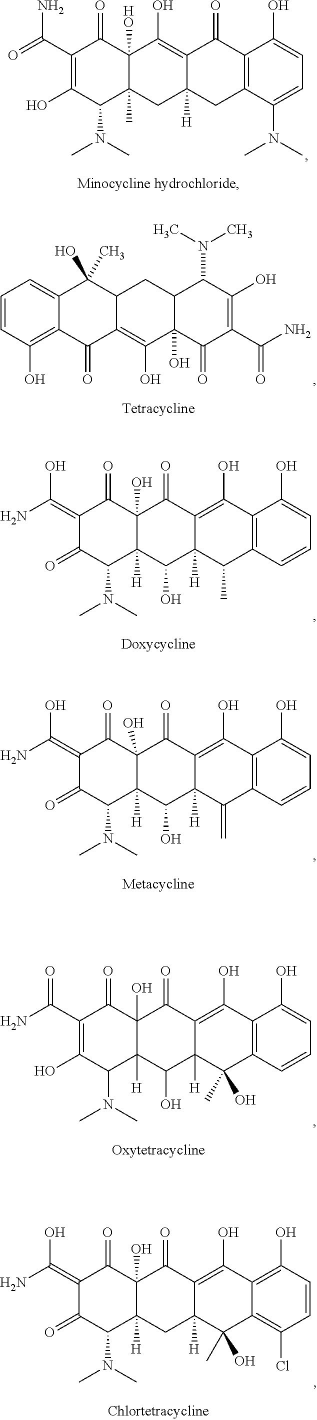 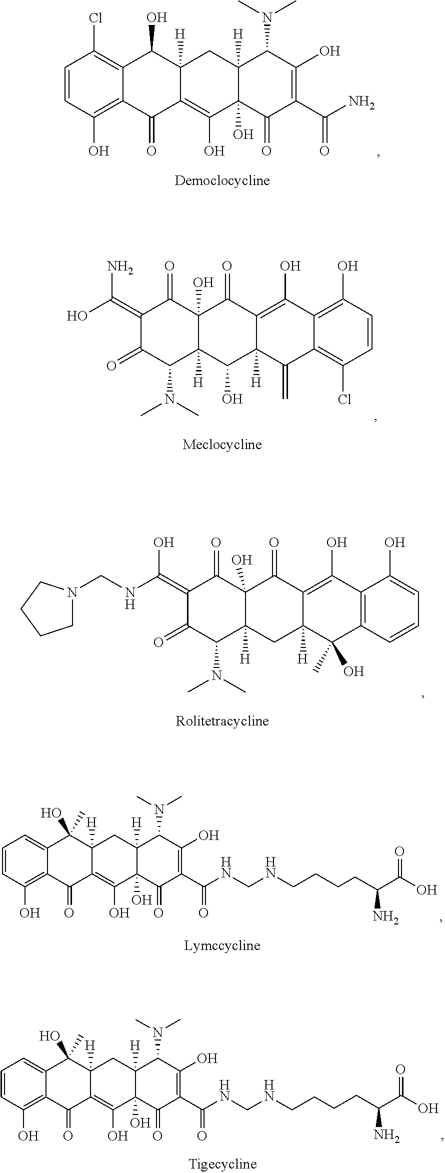
a tautomer thereof, a pharmaceutically acceptable salt thereof, a prodrug thereof, a solvate thereof, a stereoisomer thereof, an enantiomer thereof, and combinations thereof.

10. The method of claim 9, wherein the compound is Minocycline, Tetracycline or doxycycline.

11. The method of claim 1, wherein the MMP inhibitor is a diaryl ether hydroxamate having the following Formula (III) or a pharmaceutically acceptable salt, prodrug, solvate, stereoisomer or enantiomer thereof, 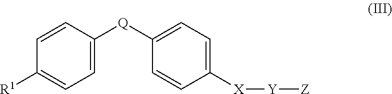
wherein
R¹ is halogen, OH, NH₂, OC₁₋₁₀alkyl unsubstituted or substituted by 1-3 halogen, or NH₂;
Q is absent or O;
X is O or S(O)₂;
Y is CH₂ or NH;
Z is C₅₋₁₄heteroaryl having 1 to 3 heteroatoms independently selected from N, O and S or 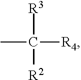
and
R², R³ and R⁴ are each independently H, C₁₋₁₀alkyl, 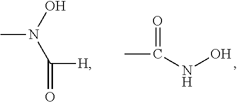
or unsubstituted or substituted C₅₋₁₄heteroaryl having 1 to 3 heteroatoms independently selected from N, O and S; or R² and R⁴ are taken together with the carbon atom to which each is attached form a 5 membered saturated heterocyclyl ring which is unsubstituted or substituted by CN or C₁₋₁₀alkyl, C₁₋₁₀alkylC₅₋₁₅aryl.

12. The method of claim 11, wherein when Q is absent, R¹ is OC(halogen)₃, X is O, Y is CH₂, Z is 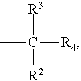
and R², R³ and R⁴ are each independently H, 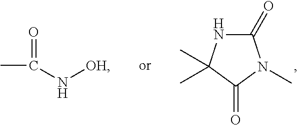
or R² and R⁴ are taken together with the carbon or nitrogen atom to form

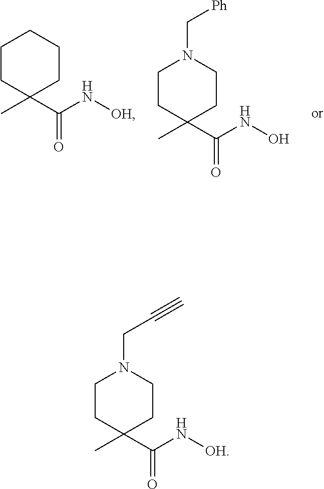

13. The method of claim 11, wherein when Q is O; R¹ is halogen or OC(halogen)₃, X is S(O)₂, and Z is

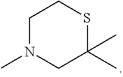

14. The method of claim 11, wherein when Q is O; R¹ is halogen or OC(halogen)₃, X is S(O)₂, Y is NH; Z is 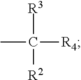
and R², R³ and R⁴ are each independently H, C₁₋₁₀alkyl,

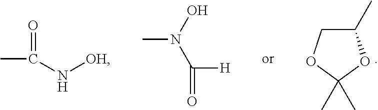

15. The method of claim 11, wherein the compound of Formula (III) is selected from the group consisting of: 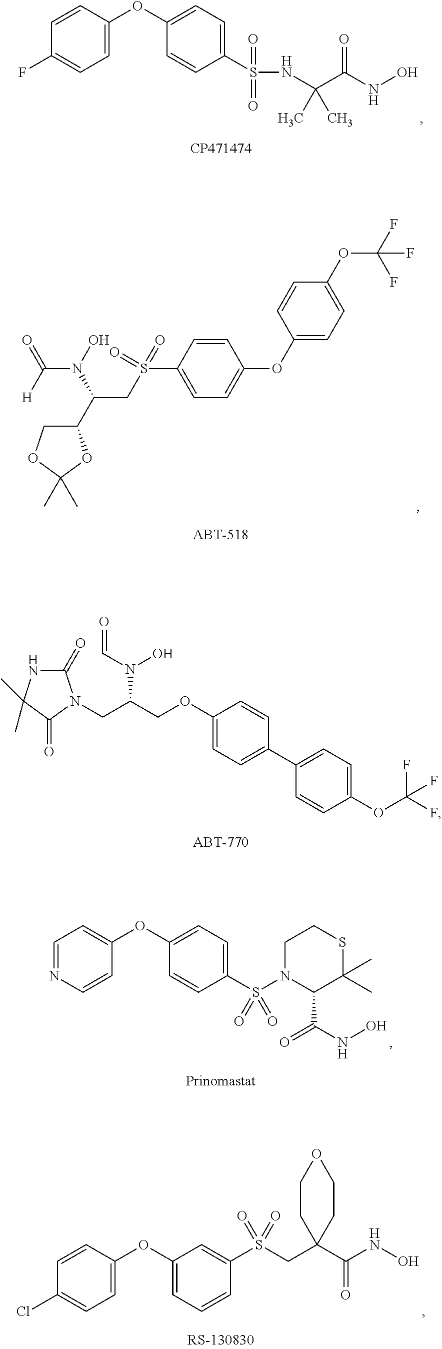 [SMILES: CC(C)(NS(=O)(=O)c1ccc(Oc2ccc(F)cc2)cc1)C(=O)NO ; CC1(C)OC[C@H]([C@@H](CS(=O)(=O)c2ccc(Oc3ccc(OC(F)(F)F)cc3)cc2)N(O)C=O)O1 ; CC1(C)NC(=O)N(C[C@@H](COc2ccc(-c3ccc(OC(F)(F)F)cc3)cc2)N(O)C=O)C1=O ; CC1(C)SCCN(S(=O)(=O)c2ccc(Oc3ccncc3)cc2)[C@H]1C(=O)NO ; O=C(NO)C1(CS(=O)(=O)c2cccc(Oc3ccc(Cl)cc3)c2)CCOCC1] 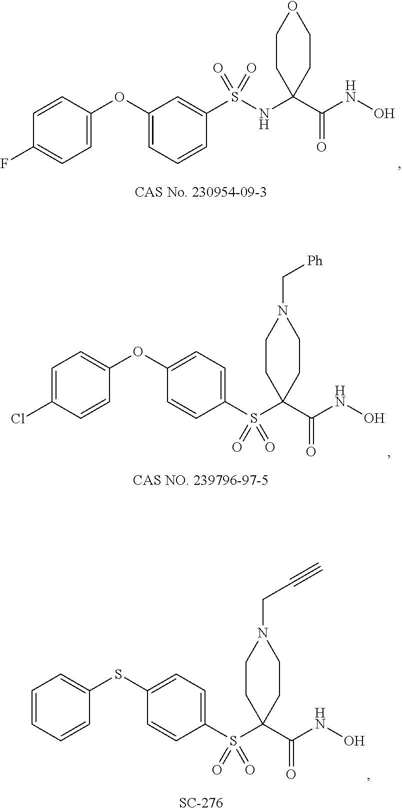 [SMILES: O=C(NO)C1(NS(=O)(=O)c2cccc(Oc3ccc(F)cc3)c2)CCOCC1 ; O=C(NO)C1(S(=O)(=O)c2ccc(Oc3ccc(Cl)cc3)cc2)CCN(Cc2ccccc2)CC1 ; C#CCN1CCC(C(=O)NO)(S(=O)(=O)c2ccc(Sc3ccccc3)cc2)CC1]
a pharmaceutically acceptable salt thereof, a prodrug thereof, a solvate thereof, a stereoisomer thereof, an enantiomer thereof, and combinations thereof.

16. The method of claim 1, wherein the MMP inhibitor is a compound having the following formula: 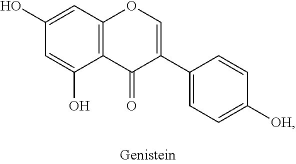 [SMILES: O=c1c(-c2ccc(O)cc2)coc2cc(O)cc(O)c12]
or a pharmaceutically acceptable salt, prodrug, solvate, stereoisomer or enantiomer thereof.

17. A method for treating a disease medicated by expression of lumican and/or collagen fibrillogenesis, and/or treating myopia and/or keratoconus disease, comprising administering to the subject a therapeutically effective amount of a TGF-beta inhibitor.

18. The method of claim 17, wherein the TGF-beta inhibitor is selected from the group consisting of: 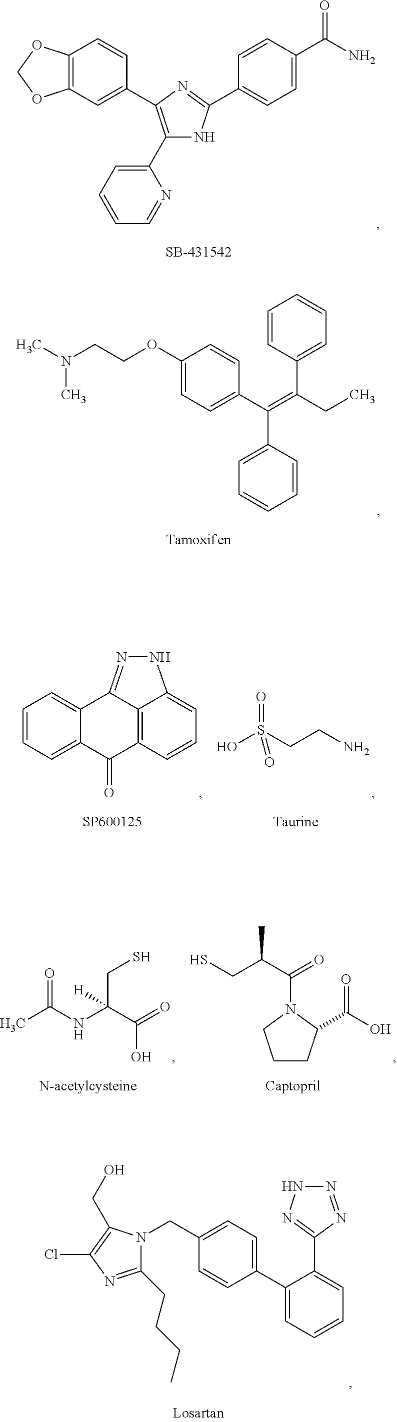 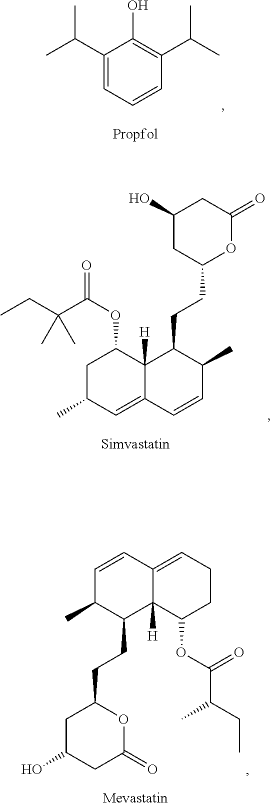
a pharmaceutically acceptable salt thereof, a prodrug thereof, a solvate thereof, a stereoisomer thereof, an enantiomer thereof, and combinations thereof.

19. The method of claim 18, wherein the TGF-beta inhibitor is Losartan, N-acetylcysteine, Propofol and Captopril.

20. The method of claim 1, wherein the method is for treating myopia.

21. The method of claim 17, wherein the method is for treating myopia.


SEQUENCE LISTING (11 sequences)

SEQ ID NO: 1 - DNA, 23 bp, Artificial Sequence
  primer
agtagaggtatttgattccggtc

SEQ ID NO: 2 - DNA, 21 bp, Artificial Sequence
  primer
gcacaagaaggtgatgaaacg

SEQ ID NO: 3 - DNA, 22 bp, Artificial Sequence
  primer
cagacttagaagtccagccaac

SEQ ID NO: 4 - DNA, 24 bp, Artificial Sequence
  primer
gcctcagagatcatctttgaatag

SEQ ID NO: 5 - DNA, 36 bp, Artificial Sequence
  primer
ggccacgcgtcgactagtacgggnngggnngggnng

SEQ ID NO: 6 - DNA, 32 bp, Artificial Sequence
  primer
cnacnacnacnaggccacgcgtcgactagtac

SEQ ID NO: 7 - DNA, 25 bp, Artificial Sequence
  morpholino antisense oligonucleotide
gatcccagagcaaacatggctgcac

SEQ ID NO: 8 - DNA, 25 bp, Artificial Sequence
  morpholino antisense oligonucleotide
cctcttacctcagttacaatttata

SEQ ID NO: 9 - DNA, 37 bp, Artificial Sequence
  primer
ataagaatgcggccgctccattaattcgacagaccag

SEQ ID NO: 10 - DNA, 37 bp, Artificial Sequence
  primer
ataagaatgcggccgcaggtagacaacacggttatgt

SEQ ID NO: 11 - DNA, 32 bp, Artificial Sequence
  primer
cgacgcgtggctgcacaacttaaattaaacct